(19) United States
(12) Patent Application Publication
(10) Pub. No.: US 2025/0277078 A1
(43) Pub. Date: Sep. 4, 2025

(54) TRI-ARM STAR BOTTLEBRUSH POLYMER WITH DEFINED VISCOSITY AND OPTICAL PROPERTIES FOR USE IN A NOVEL INTRAOCULAR LENS

(71) Applicants: DUKE UNIVERSITY, Durham, NH (US); ADAPTILENS, LLC, Chestnut Hill, MA (US)

(72) Inventors: Matthew L. BECKER, Chapel Hill, NC (US); Metin KARAYILAN, Durham, NC (US); Liane CLAMEN, Chestnut Hill,, MA (US)

(73) Assignees: DUKE UNIVERSITY, Durham, NC (US); ADAPTILENS, LLC, Chestnut Hill, MA (US)

(21) Appl. No.: 18/866,289

(22) PCT Filed: May 19, 2023
(86) PCT No.: PCT/US2023/022953

§ 371 Date: Nov. 15, 2024

Related U.S. Application Data
(60) Provisional application No. 63/344,186, filed on May 20, 2022.

(51) Int. Cl. C08F 293/00; A61L 27/18; A61L 27/52; B29D 11/00; B29K 83/00

(52) U.S. Cl. CPC C08F 293/005; A61L 27/18; A61L 27/52; A61L 2430/16; B29D 11/00442; B29K 2083/00; C08F 2438/03; C08F 2810/20

(57) ABSTRACT

The present invention provides a tri-arm star bottlebrush polymer or copolymer that can be photocrosslinked to form a solvent free soft and optically clear elastomeric gel with a specific refractive index and Young's Modulus, making it suitable for implantation and for use as an accommodating intraocular lens (IOL), a pseudoaccomodating, presbyopia correcting IOL, or a custom-molded IOL. In various embodiments, the tri-arm star bottlebrush polymer is formed using a trifunctional reversible addition fragmentation chain-transfer (RAFT) agent and will have three methacrylate and/or acrylate polymer chains extending therefrom. These methacrylate polymer chains comprising the polymerized residues of a methacrylate macromonomer and one or more hydroxy-functionalized methacrylate chain extenders with alkene functional groups covalently bonded thereto. In some of these embodiments, the thiol containing end groups of the trifunctional RAFT agent, if any, are removed using a thermally or chemically activated radical generating compound to produce an optically clear polymer.

### CROSS REFERENCE TO RELATED APPLICATIONS

This application claims the benefit of U.S. provisional patent application Ser. No. 63/344,186 entitled “Tri-Arm Star Bottlebrush Polymer with Defined Viscosity and Optical Properties for Use in a Novel Intraocular Lens,” filed May 20, 2022, and incorporated herein by reference in its entirety.

### NAMES OF THE PARTIES TO A JOINT RESEARCH AGREEMENT

The present application stems from work done pursuant to a Joint Research Agreement between Duke University of Durham, North Carolina and Adaptilens, LLC of Chestnut Hill, Massachusetts.

### FIELD OF THE INVENTION

One or more embodiments of the present invention relate to bottlebrush polymers. In certain embodiments, the invention relates to biostable polymer bottlebrushes that have tunable viscosity and optical properties for use in intraocular lenses.

### BACKGROUND OF THE INVENTION

Polymers are split into two general classes: thermoplastics and thermosets. Due to lacking a crosslinked network, thermoplastics can be soluble in good solvents and become soft or melt when heated, in this way they can be reprocessable and remoldable. Thermosets, on the other hand, contain crosslinked networks and are irreversibly cured for high-performance applications. Elastomers are a class of materials that contain lightly crosslinked polymer networks which give elasticity to the elastomers. Soft elastomers can be prepared by increasing the molecular weight of the network strand (polymer chains between two junctions/crosslinking points) and by decreasing the chain entanglement of the polymer chains. Polymer chains start generating entanglement within the system above a certain molecular weight called chain entanglement molecular weight, and these entangled chains are permanently trapped upon crosslinking which then behave as topological crosslinks. These chain entanglements can be prevented or delayed by changing the “volume” of the polymer chain which is possible through changing the architecture of the polymer chain from linear to branched or regime where the side chains are highly stretched and crowded which forces the backbone into a highly extended state, commonly referred to as bottlebrush.

Bottlebrush polymers (BBPs) are a type of polymers with long and densely grafted side chains. BBPs can be synthesized using different approaches such as grafting to, grafting from, and grafting through approaches. In the grafting to approach, long polymer chains asymmetrically terminated with a functional group can be chemically connected to a polymer backbone with many functional groups ideally on every repeating unit which are reactive to the functional group on the long polymer chains. The grafting from approach requires a polymer backbone with initiator sites ideally on every repeating unit. Using small-molecule monomers, polymer side chains can be grown from the polymer backbone, typically using Controlled Radical Polymerization (CRP) techniques such as Atom Transfer Radical Polymerization (ATRP) and Reversible Addition-Fragmentation Chain Transfer (RAFT) Polymerization. Finally, the grafting through approach allows using macromonomers with a polymerizable unit on one chain end and utilizing different polymerization techniques such as reversible deactivation radical polymerization (RDRP; i.e., ATRP or RAFT) or ring-opening metathesis polymerization (ROMP). While RDRP requires commonly used styrenic or (meth)acrylic functional groups, ROMP utilizes norbornene-based polymer chain ends to polymerize macromonomers into BBPs.

The different approaches discussed earlier have given rise to different grafting densities. Generally, the grafting to approach gives the lowest grafting density and the grafting through approach produces the highest grafting density among these three approaches. The difference in the grafting density due to the selection of different grafting approaches stems from the steric hindrance generated between the components (i.e., between polymer backbone-long side chain for grafting to, growing chains on the polymer backbone for grafting from, and growing BBPs-macromonomers for grafting through). To synthesize true BBPs, the grafting density should be high enough that it gives the BBPs rigidity and prevents entanglement. BBPs and bottlebrush gels can be differentiated depending on the macromonomer identity (the side chain plays a central role to determine the final properties of the resulting BBPs), side-chain degree of polymerization (DP), backbone DP, grafting density (distance between each side chain), and crosslinking density (for bottlebrush gels).

Recent developments in the technology of certain intraocular lenses (IOLs) and accommodating or adaptive intraocular lenses (A-IOLs) for use in surgery for treating cataracts have created a need for optically clear, biostable, polymer filling materials having a suitable refractive index (nᵣ) and complex viscosity. Over time, the lens in the eye becomes stiffer and less flexible, making it more difficult for the eye to focus on near objects. This gradual, age-related loss of accommodation is called presbyopia. As people age further, the lens becomes thickened and opaque forming a cataract and causing blurred vision. The standard of care is to have cataract surgery to remove the cataract and replace it with an IOL. The current standard lenses are monofocal lenses which cannot adjust for both near and far sight, leaving patients dependent upon glasses. A-IOLs would allow patients to see clearly over a range of distances without eyeglasses or contact lenses. Various different polymers have been used in lens refilling TOLs and in other types of A-IOLs with mixed results. Many of these materials require solvents or other diluting fluids to arrive at working viscosities. Both Jean Marie Parel, MD (Bascom Palmer) and Steven Koopmans (Pharmacia) attempted to restore accommodation by refilling the capsular bag with a soft polymer (Hao et al, 2010; Koopmans, 2003 and 2006). They both injected in situ polymerizing materials directly into the capsular bag. Both scientists ended their efforts after in vivo animal trials presented significant complications including inflammation of the eye and posterior capsular opacification (PCO). (Koopmans, 2014, Hao 2012) Similarly, Nishi's attempts at developing an endocapsular balloon filled with silicone oil failed when severe posterior capsule opacification occurred (Nishi 1997) The Fluid Vision IOL is a hydrophobic acrylic lens with a hollow optic and two hollow haptics filled with silicone oil. When the ciliary muscles contract, the oil shifts from the haptics into the optic to change the shape of the lens. Although this IOL is still in clinical trials, problems with the lens include the slow speed at which patients focus and the inconsistent effective lens position. (Young)

What is needed in the art is a clear, synthetic route to generating optically clear, biostable polymer brushes that have tunable viscosity and optical properties that can be used as a soft, flexible material to be molded into A-IOLs. This polymer could be used to create a soft, flexible accommodating IOL. The A-IOL would remain soft and flexible, like the young, healthy human lens. When the ciliary muscles of the eye contract during accommodation, the flexible lens will change shape such that the power of the lens will increase and allow the patient to focus at near. Once the muscles of accommodation relax, the lens will resume its baseline shape, allowing the patient to see at distance. Alternatively, this novel material could be used as the optic of a presbyopia correcting intraocular lens (IOL).

Presbyopia correcting IOLs include multifocal, Trifocal and Extended Depth of Focus (EDOF) IOLs. These IOLs correct for presbyopia, but are pseudoaccomodating, meaning they do not work with the ciliary muscles and other accommodating structures inside the eye. Rather, they are flat, and allow for an increased range of vision by providing multiple focal points. Multifocal IOLs can be refractive, diffractive or EDOF. Refractive multifocals have concentric zones of power, diffractive multifocals utilize concentric diffractive surfaces to help reduce glare and higher-order aberrations. (https://www.reviewofoptometry.com/article/four-steps-to-make-premium-iols-worth-the-cost) Unfortunately, both refractive and diffractive surfaces cause the in-focus image to be overlaid by at least one out-of-focus image, creating problems such as haloes, glare, and decreased contrast sensitivity. EDOF IOLs have a unique diffractive pattern combined with a technology correcting for corneal chromatic aberration. While this provides good depth of focus with good contrast sensitivity, EDOF IOLs provide worse near vision than diffractive IOLs. The problems with current presbyopia correcting IOLs include reduced quality of vision, decreased contrast sensitivity and negative side effects such as photopisas (https://millennialeye.com/articles/2016-jul-aug/night-vision-and-presbyopia-correcting-iols/) To address these problems, this novel material could be used as the optic of a presbyopia correcting intraocular lens (IOL). This novel material is softer than the material currently used in optics of presbyopia correcting IOLs and could allow for improvements in the optics of these premium IOLs. Alternatively, this novel material could be used in an IOL that may or may not be an A-IOL or a presbyopia correcting IOL, but is a custom-made IOL.

Custom-made IOLs do not exist. Before cataract surgery, three primary measurements of the eye are taken and inserted into a formula to determine the correct IOL power for each eye. These measurements are the axial length of the eye, the corneal curvature (keratometry), and the anterior chamber depth. Currently, these three measurements are used only to determine the IOL power for each patient's eye. However, if lenses could be molded and custom-made for each eye, these measurements would be considered alongside other measurements of a patient's eye in order to mold a unique accommodating intraocular lens so that each eye could see optimally.

Since custom-made IOLs do not exist, there are certain ocular problems that cannot be corrected by current IOLs. For example, toric IOLs can only correct “regular” astigmatism. Regular astigmatism is defined as a symmetrical steepening along a specific axis, bisecting in the center of the cornea in the configuration of a bowtie. In contrast, irregular astigmatism is uneven, or curved in multiple directions. Irregular astigmatism, often referred to as a Complex Cornea, is much more difficult to treat and cannot be treated with current IOLs.

Similarly, while lower order aberrations of the cornea (such as sphere and cylinder) can be corrected with glasses or contact lenses, many higher order aberrations (HOA) of the cornea (such as coma and trefoil) cannot be corrected. More than 60 different HOAs have been identified, and many cannot be corrected with glasses or contact lenses.

There is a need in the art for a solvent-free, polymer capable of forming synthetic elastomer that is capable of being molded in a custom-mold for each patient, allowing for the creation of an entirely new class of IOLs: custom-made IOLs.

### SUMMARY OF THE INVENTION

In one or more embodiments, the present invention provides a tri-arm star bottlebrush polymer or copolymer that can be photocrosslinked to form a solvent free soft elastomeric gel suitable for implantation as an A-IOL, a pseudoaccommodive presbyopia correcting IOL, or as a custom-molded artificial intraocular lens (IOL) for use in treating cataracts. In various embodiments, tri-arm star bottlebrush polymer is formed using a trifunctional reversible addition fragmentation chain-transfer (RAFT) agent and will have three methacrylate and/or acrylate polymer chains extending therefrom. Each of these methacrylate polymer chains comprising the polymerized residues of a methacrylate macromonomer, such as PDMS-MA, and the residues of one or more hydroxy-functionalized methacrylate chain extenders, such as 2-hydroxyethyl methylacrylate (HEMA), and will have a plurality of alkene functional groups covalently bonded to the methacrylate polymer chains through terminal hydroxyl groups on methacrylate chain extenders. In some of these embodiments, the thiol containing end groups of the trifunctional RAFT agent, if any, are removed using a thermally or chemically activated radical generating compound, such as 2,2′-azobis(2-methylpropionitrile) (AIBN) to produce an optically clear polymer. The resulting polymers are then used to form a resin and photocrosslinked to for a soft, flexible and optically clear elastomer having a Young's modulus of from about 0.005 MPa to about 0.05 MPa and an ultimate compressive strength (UCS) of from about 0.002 MPa to about 0.5 MPa. In various embodiments, molds are used to form accommodating IOLs of various powers. In other embodiments, this material is used in pseudoaccommodative, presbyopia correcting IOLs such as multifocal, trifocal or EDOF IOLs. In other embodiments, custom made molds are used to form custom-molded artificial intraocular lenses (IOLs) designed to address the needs of a specific patient.

In a first aspect, the present invention is directed to a tri-arm star bottlebrush polymer for use in an intraocular lens comprising the residue of a trifunctional reversible addition fragmentation chain-transfer (RAFT) agent and three acrylate or, preferably, methacrylate polymer chains extending therefrom, wherein each one of the three methacrylate polymer chains comprising the polymerized residues of two or more acrylate or methacrylate macromonomers. In one or more embodiment, the trifunctional RAFT agent comprises three arms each having at least one site capable of RAFT polymerization, wherein each arm further comprises a sulfur containing end group. In some embodiments, the trifunctional RAFT agent comprises 1,1,1-tris [(dodecylthiocarbonothioylthio)-2-methylpropionate]ethane (Tris (DDMAT).

In one or more embodiments, the tri-arm star bottlebrush polymer of the present invention includes any one or more of the above referenced embodiments of the first aspect of the present invention wherein the two or more methacrylate macromonomers comprise monomethacryloxypropyl terminated polydimethylsiloxane-asymmetric (PDMS-MA). In one or more embodiments, the tri-arm star bottlebrush polymer of the present invention includes any one or more of the above referenced embodiments of the first aspect of the present invention wherein the methacrylate macromonomers have the formula:

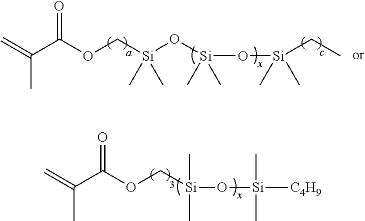

where a is an integer from about 1 to about 6; and x is an integer from about 2 to about 30. In one or more embodiments, the tri-arm star bottlebrush polymer of the present invention includes any one or more of the above referenced embodiments of the first aspect of the present invention wherein the PDMS-MA has a mass average molecular weight from about 600 kDa to about 800 kDa.

In one or more embodiments, the tri-arm star bottlebrush polymer of the present invention includes any one or more of the above referenced embodiments of the first aspect of the present invention having the formula:

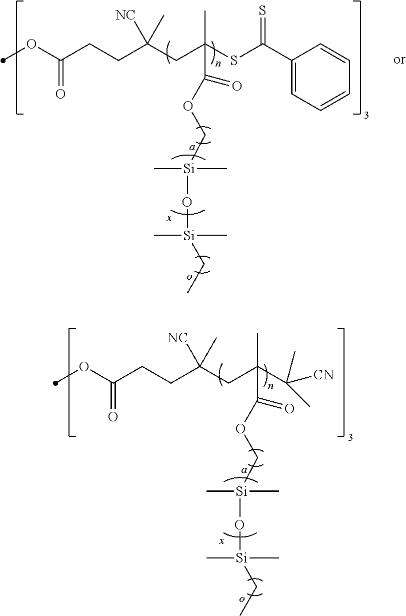

where a is an integer from about 1 to about 6; c is an integer from 1 to 5; x is an integer from about 2 to about 20 and n is an integer from about 10 to about 80.

In one or more embodiments, the tri-arm star bottlebrush polymer of the present invention includes any one or more of the above referenced embodiments of the first aspect of the present invention having the formula:

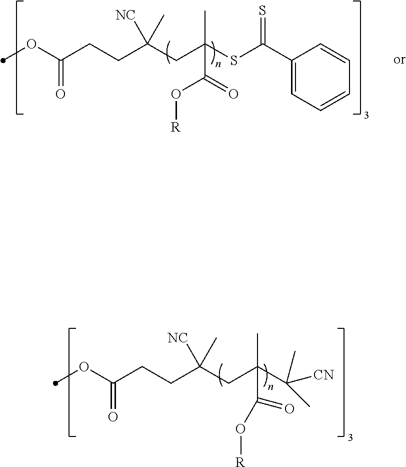

wherein R has the formula

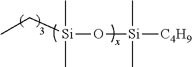

where x is an integer from about 5 to about 10; and n is an integer from about 10 to about 80. In one or more embodiments, the tri-arm star bottlebrush polymer of the present invention includes any one or more of the above referenced embodiments of the first aspect of the present invention wherein each of the three methacrylate polymer chains further comprises the residues of one or more hydroxy-functionalized methacrylate chain extenders. In some embodiments, the one or more hydroxy-functionalized methacrylate chain extenders is 2-hydroxyethyl methylacrylate (HEMA) molecules.

In one or more embodiments, the tri-arm star bottlebrush polymer of the present invention includes any one or more of the above referenced embodiments of the first aspect of the present invention having a formula selected from:

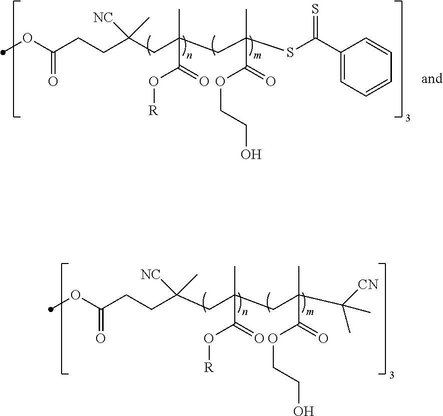

wherein R has the formula

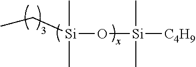

where x is an integer from about 5 to about 10; n is a mole percent from about 80% to about 99%; and m is a mole percent from about 1% to about 20%.

In one or more embodiments, the tri-arm star bottlebrush polymer of the present invention includes any one or more of the above referenced embodiments of the first aspect of the present invention wherein n is a mole percent from about 90% to about 99%. In one or more embodiments, the tri-arm star bottlebrush polymer of the present invention includes any one or more of the above referenced embodiments of the first aspect of the present invention wherein n is a mole percent from about 95% to about 99%. In one or more embodiments, the tri-arm star bottlebrush polymer of the present invention includes any one or more of the above referenced embodiments of the first aspect of the present invention wherein each of the three methacrylate polymer chains comprises an A: B block copolymer having a poly(monomethacryloxypropyl terminated polydimethylsiloxane) A block and a poly(2-hydroxyethyl methylacrylate) B block.

In one or more embodiments, the tri-arm star bottlebrush polymer of the present invention includes any one or more of the above referenced embodiments of the first aspect of the present invention further comprising a plurality of alkene functional groups covalently bonded to the three methacrylate polymer chains through terminal hydroxyl groups on the two or more hydroxy-functionalized methacrylate chain extenders. In one or more embodiments, the tri-arm star bottlebrush polymer of the present invention includes any one or more of the above referenced embodiments of the first aspect of the present invention further comprising a plurality of alkene functional groups covalently bonded to the three methacrylate polymer chains through terminal hydroxyl groups on the two or more 2-hydroxyethyl methylacrylate (HEMA) molecules.

In one or more embodiments, the tri-arm star bottlebrush polymer of the present invention includes any one or more of the above referenced embodiments of the first aspect of the present invention having a formula selected from:

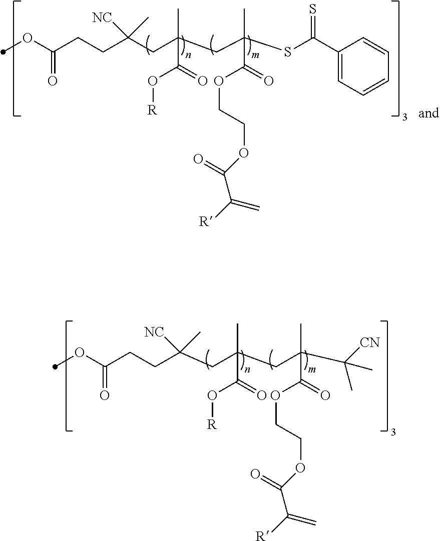

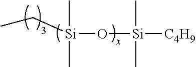

wherein R has the formula where x is an integer from about 5 to about 10; R′ is H or CH₃; n is a mole percent from about 80% to about 99%; and m is a mole percent from about 1% to about 20%.

In one or more embodiments, the tri-arm star bottlebrush polymer of the present invention includes any one or more of the above referenced embodiments of the first aspect of the present invention wherein any of the sulfur containing end groups remaining from the trifunctional a reversible addition fragmentation chain-transfer (RAFT) agent after polymerization have been removed. In one or more embodiments, the tri-arm star bottlebrush polymer of the present invention includes any one or more of the above referenced embodiments of the first aspect of the present invention wherein any sulfur containing end groups remaining from the trifunctional a reversible addition fragmentation chain-transfer (RAFT) agent after polymerization have been removed. In one or more embodiments, the tri-arm star bottlebrush polymer of the present invention includes any one or more of the above referenced embodiments of the first aspect of the present invention wherein any of the sulfur containing end groups remaining from the trifunctional a reversible addition fragmentation chain-transfer (RAFT) agent have been removed.

In one or more embodiments, the tri-arm star bottlebrush polymer of the present invention includes any one or more of the above referenced embodiments of the first aspect of the present invention having a formula selected from:

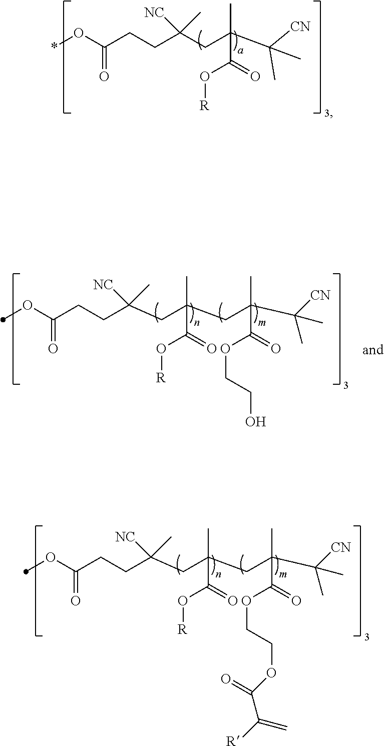

wherein R has the formula

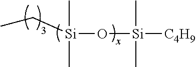

where x is an integer from about 5 to about 10; R′ is H or CH₃; a is an integer from about 10 to about 80; n is a mole percent from about 80% to about 99%; and m is a mole percent from about 1% to about 20%.

In one or more embodiments, the tri-arm star bottlebrush polymer of the present invention includes any one or more of the above referenced embodiments of the first aspect of the present invention wherein the tri-arm star bottlebrush polymer is optically clear. In one or more embodiments, the tri-arm star bottlebrush polymer of the present invention includes any one or more of the above referenced embodiments of the first aspect of the present invention wherein the tri-arm star bottlebrush polymer has a refractive index of from about 1.40 to about 1.49, preferably from about 1.42 to about 1.48, and more preferably from about 1.43 to about 1.46 at 37° C. In one or more embodiments, the tri-arm star bottlebrush polymer of the present invention includes any one or more of the above referenced embodiments of the first aspect of the present invention having a degree of polymerization for each arm between about 10 and about 80.

This polymer could be used to create a soft, flexible accommodating IOL. The A-IOL would remain soft and flexible like the young, healthy human lens. When the ciliary muscles of the eye contract during accommodation, the flexible lens will change shape such that the power of the lens will increase and allow the patient to focus at near. Once the muscles of accommodation relax, the lens will resume its baseline shape, allowing the patient to see at distance. Alternatively, this novel material could be used as the optic of a presbyopia correcting intraocular lens (IOL).

Presbyopia correcting IOLs include multifocal, Trifocal and Extended Depth of Focus (EDOF) IOLs. These IOLs correct for presbyopia, but are pseudoaccomodating, meaning they do not work with the ciliary muscles and other accommodating structures inside the eye. Rather, they are flat, and allow for an increased range of vision by providing multiple focal points. Multifocal IOLs can be refractive, diffractive or EDOF. This novel material is softer than the material currently used in optics of presbyopia correcting IOLs and could allow for improvements in the optics of these premium IOLs. Alternatively, this novel material could be used in an IOL that may or may not be an A-IOL or a presbyopia correcting IOL, but is a custom-made IOL.

While lower order aberrations of the cornea (such as sphere and cylinder) can be corrected with glasses or contact lenses, many higher order aberrations (HOA) of the cornea (such as coma and trefoil) cannot be corrected. More than 60 different HOAs have been identified, and many cannot be corrected with glasses or contact lenses. With a custom-made IOL, preoperative measurements of the eye can be used to determine an ideal IOL to correct for a specific eye's needs. Using corneal topography, aberrometry (wavefront technology), and other imaging and diagnostic tools, one could create a computer model of the ideal shape of an IOL or an accommodating IOL for each patient. Using the computer model, a mold could be made, and the Generation 2 material could be molded inside of it to create a custom-made IOL to correct for each specific eye's needs.

There is a need in the art for a solvent-free, polymer capable of forming synthetic elastomer that is capable of being molded in a custom-mold for each patient, allowing for the creation of an entirely new class of IOLs: custom-made IOLs.

In a second aspect, the present invention is directed to a photocurable tri-arm star bottlebrush polymer resin comprising the tri-arm star bottlebrush polymer described above, dimethacryloxypropyl terminated polydimethylsiloxane (PDMS-diMA) and a photoinitiator. In some of these embodiments, the photocurable tri-arm star bottlebrush polymer resin comprises from about 2% to about 98% PDMS-diMA by volume. In one or more embodiments, the photocurable tri-arm star bottlebrush polymer resin of the present invention includes any one or more of the above referenced embodiments of the second aspect of the present invention wherein the photoinitiator is 2,2-dimethoxy-1,2-diphenylethanone. In one or more embodiments, the photocurable tri-arm star bottlebrush polymer resin of the present invention includes any one or more of the above referenced embodiments of the second aspect of the present invention wherein the resin is optically clear.

In a third aspect, the present invention is directed to a soft and flexible tri-arm star bottlebrush hydrogel network for use in artificial intraocular lenses comprising the photocurable tri-arm star bottlebrush polymer resin described above. In one or more embodiments, the soft and flexible photocured tri-arm star bottlebrush hydrogel network of the present invention includes any one or more of the above referenced embodiments of the third aspect of the present invention having Young's modulus of from about 0.005 MPa to about 0.05 MPa. In one or more embodiments, the soft and flexible photocured tri-arm star bottlebrush hydrogel network of the present invention includes any one or more of the above referenced embodiments. In one or more embodiments, the soft and flexible photocured tri-arm star bottlebrush hydrogel network of the present invention includes any one or more of the above referenced embodiments of the third aspect of the present invention having an ultimate compressive strength (UCS) of from about 0.002 MPa to about 0.5 MPa. In one or more embodiments, the soft and flexible photocured tri-arm star bottlebrush hydrogel network of the present invention includes any one or more of the above referenced embodiments of the third aspect of the present invention wherein the hydrogel network is optically clear.

In a fourth aspect, the present invention is directed to an artificial intraocular lens comprising the tri-arm star bottlebrush polymer described herein. In some of these embodiments, artificial intraocular lens has a Young's modulus of from about 0.005 MPa to about 0.05 MPa. In some of these embodiments, artificial intraocular lens has an ultimate compressive strength (UCS) of from about 0.002 MPa to about 0.5 MPa after curing with ultraviolet light. In these embodiments, the artificial intraocular lens is optically clear.

In a fifth aspect, the present invention is directed to a method for making a photocured tri-arm star bottlebrush hydrogel network comprising: combining a tri-arm star bottlebrush polymer described herein with a bis-methacryl terminated polydimethylsiloxane crosslinker, and a photoinitiator to form an uncured tri-arm star bottlebrush polymer resin; and exposing the uncured tri-arm star bottlebrush polymer resin to ultraviolet light to produce a photocured tri-arm star bottlebrush hydrogel network. In one or more embodiments, the method for making a photocured tri-arm star bottlebrush hydrogel network of the present invention includes any one or more of the above referenced embodiments of the fifth aspect of the present invention wherein the uncured tri-arm star bottlebrush polymer resin comprises from about 2% to about 98% by volume PDMS-diMA. In one or more embodiments, the method for making a photocured tri-arm star bottlebrush hydrogel network of the present invention includes any one or more of the above referenced embodiments of the fifth aspect of the present invention wherein the photoinitiator is 2,2-dimethoxy-1,2-diphenylethanone.

In one or more embodiments, the method for making a photocured tri-arm star bottlebrush hydrogel network of the present invention includes any one or more of the above referenced embodiments of the fifth aspect of the present invention wherein the photocured tri-arm star bottlebrush hydrogel network has a Young's modulus of from about 0.005 MPa to about 0.05 MPa. In one or more embodiments, the method for making a photocured tri-arm star bottlebrush hydrogel network of the present invention includes any one or more of the above referenced embodiments of the fifth aspect of the present invention wherein the photocured tri-arm star bottlebrush hydrogel network has an ultimate compressive strength (UCS) of from about 0.002 MPa to about 0.5 MPa. In one or more embodiments, the method for making a photocured tri-arm star bottlebrush hydrogel network of the present invention includes any one or more of the above referenced embodiments of the fifth aspect of the present invention wherein the photocured tri-arm star bottlebrush hydrogel network produced is optically clear.

In a another aspect, the present invention is directed to a method of making an artificial intraocular lens comprising the tri-arm star bottlebrush polymer described herein comprising: preparing a mold shaped to hold an artificial intraocular lens of a desired size and shape; combining a tri-arm star bottlebrush polymer with PDMS-diMA, and a photoinitiator to form an uncured tri-arm star bottlebrush polymer resin; filling the mold with the uncured tri-arm star bottlebrush polymer resin; and exposing the uncured tri-arm star bottlebrush polymer resin to ultraviolet light to produce an artificial intraocular lens by crosslinking the uncured tri-arm star bottlebrush polymer resin to form an optically clear photocured tri-arm star bottlebrush hydrogel network.

In a yet another aspect, the present invention is directed to a method of making an artificial intraocular lens comprising the tri-arm star bottlebrush polymer described herein comprising: creating a computer model of the ideal shape of an accommodative IOL for a patient; using the computer model to generate a mold shaped to the ideal shape; combining a tri-arm star bottlebrush polymer with PDMS-diMA, and a photoinitiator to form an uncured tri-arm star bottlebrush polymer resin; filling the mold with the uncured tri-arm star bottlebrush polymer resin; and then exposing the uncured tri-arm star bottlebrush polymer resin to ultraviolet light to crosslink it and produce an artificial intraocular lens having an ideal shape for the patient and comprising photocured tri-arm star bottlebrush hydrogel network.

These and other features of the systems and methods of the subject invention will become more readily apparent to those skilled in the art from the following detailed description of the preferred embodiments taken in conjunction with the drawings.

### BRIEF DESCRIPTION OF THE DRAWINGS

For a more complete understanding of the features and advantages of the present invention, reference is now made to the detailed description of the invention along with the accompanying figures in which:

FIG. 1A is a reaction scheme for forming a tri-arm star bottlebrush polymer according to one or more embodiments of the present invention.

FIG. 1B is a reaction scheme for forming a tri-arm star bottlebrush hydrogel network according to one or more embodiments of the present invention.

FIG. 2 is a ¹H NMR spectrum of poly (PDMS-MA) tri-arm star bottlebrush polymer in CDCl₃. * Residual solvent impurities.

FIG. 3 is a ¹H NMR spectrum of HEMA chain-extended poly(PDMS-MA-co-HEMA) tri-arm star polymer in CDCl3. * Residual solvent impurities.

FIG. 4 is a ¹H NMR spectrum of an end-group functionalized tri-arm star bottlebrush polymer in CDCl₃.

FIGS. 5A-C are schemes and images regarding curing tri-arm star polymer resin under UV irradiation using a Teflon mold wherein FIG. 5A shows the process fur curing the resin, FIG. 5B is an image showing the formed IOLs, and FIG. 5C is an image showing the TEFLON™ mold used to form the IOLs shown in FIG. 5B.

FIG. 6 in an infrared spectrum of cured polymer resin to show all the methacrylic functional groups being consumed.

FIG. 7 is a digital image of a compression test performed on a disc shape elastomer.

FIGS. 8A-H are stress-strain curves of some of the elastomers on Table 1. Dimension of the discs: height (mm)=2.6 (FIG. 8A), 3.1 (FIG. 8B), 4.4 (FIG. 8C), 4.4 (FIG. 8D), 5.5 (FIG. 8E), 4.3 (FIG. 8F), 4.4 (FIG. 8G), and 4.9 (FIG. 8F); diameter=7.8 mm for all the discs.

### DETAILED DESCRIPTION OF THE ILLUSTRATIVE EMBODIMENTS

The following is a detailed description of the disclosure provided to aid those skilled in the art in practicing the present disclosure. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description of the disclosure herein is for describing particular embodiments only and is not intended to be limiting of the disclosure.

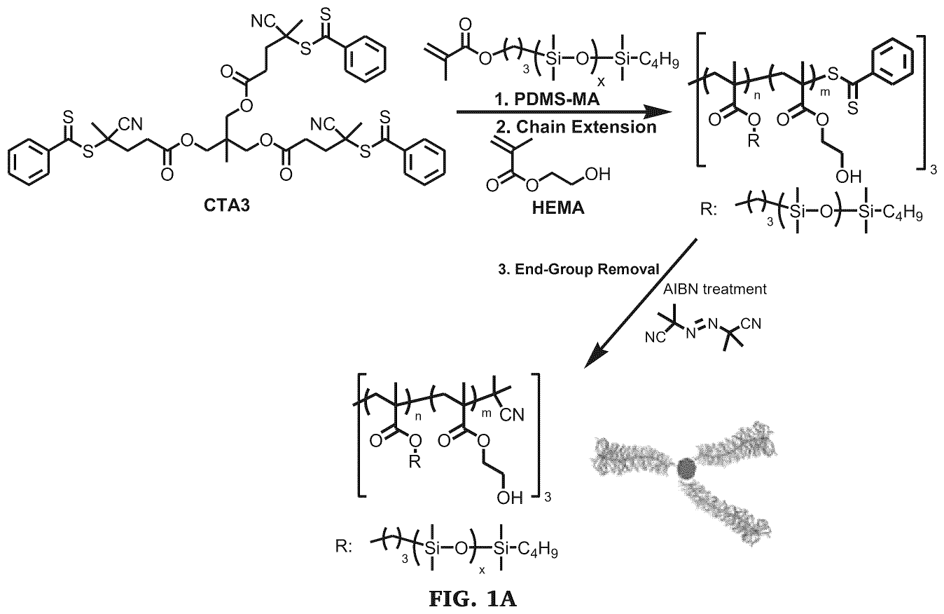

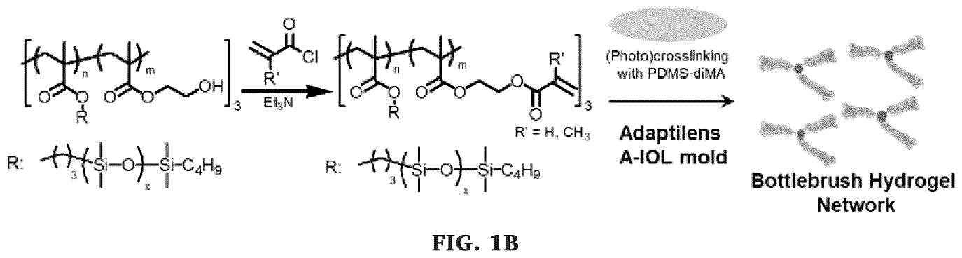

As set forth above, the present invention provides a tri-arm star bottlebrush polymer or copolymer that can be photocrosslinked to form a solvent free soft elastomeric gel suitable for implantation and molded accommodating intraocular lenses (A-IOLs), presbyopia correcting IOLs, or custom-molded artificial intraocular lenses (IOLs) for use in treating cataracts. As can be seen in FIG. 1A, the tri-arm star bottlebrush polymer is formed using a trifunctional reversible addition fragmentation chain-transfer (RAFT) agent and will have three methacrylate and/or acrylate polymer chains extending therefrom.

As can be seen, each of these methacrylate polymer chains comprising the polymerized residues of a methacrylate macromonomer, such as PDMS-MA, and the residues of one or more hydroxy-functionalized methacrylate chain extenders, such as 2-hydroxyethyl methylacrylate (HEMA), and will have a plurality of alkene functional groups covalently bonded to the methacrylate polymer chains through terminal hydroxyl groups on methacrylate chain extenders. In some of these embodiments, the thiol containing end groups of the trifunctional RAFT agent, if any, are removed using a thermally or chemically activated radical generating compound, such as 2,2′-azobis(2-methylpropionitrile) (AIBN) to produce an optically clear polymer as shown.

As can be seen in FIG. 1B, the resulting polymers are then used to form a resin and photocrosslinked to form a soft, flexible, and optically clear elastomer. In various embodiments, this optically clear elastomer will have a Young's modulus of from about 0.005 MPa to about 0.05 MPa and an ultimate compressive strength (UCS) of from about 0.002 MPa to about 0.5 MPa. In the embodiment shown in FIG. 1B, some of the HEMA groups present in the bottle brush precursors are derivatized with methacryloyl chloride to facilitate radical crosslinking with 3-arm methacrylate groups. As will be apparent, the gel formation can be initiated using an appropriate mold coupled with thermal or photochemical conditions. In various embodiments, custom made molds are used to form custom-molded artificial intraocular lenses (IOLs) designed to address the needs of a specific patient.

While it was suspected that the three-arm bottlebrush polymer networks of the present invention would likely be tougher and more tolerant to defects than a linear gel, it was surprising to find that these polymer networks formed an optically clear solvent free soft gel suitable for implantation and having a tunable refractive index making them ideally suited for artificial IOL applications. Unlike some prior art systems, these artificial IOLs have the advantage of being are self-contained, without a need for a shell. In some embodiments, these three-arm bottlebrush polymer networks will also incorporate UV chromophores for UV light filtration.

Further, the accommodating IOLs of the present invention are advantageous because compared to other devices, they utilize natural accommodation to vary precisely the optical power of the eye without damaging the tissue thereof, or the circulating aqueous materials. In a preferred embodiment the IOL is soft and flexible to ensure the IOL-eye system re-establishes the accommodative mechanism so that the optical system of the patient can respond to changes in spatial images and illumination; permitting the lens to be installed by a simple procedure that can be quickly performed. In addition, the IOL localizes in the natural capsule so as to minimize de-centering and accommodation loss; providing functional performance similar to a natural eye; and allowing volumetric accommodation so that the ciliary muscle can control accommodation of the IOL. As a result, a greater variety of patients with lens disease can be provided with natural, responsive acuity, under a greater variety of circumstances, including but not limited to, enhanced capacity for accommodation, reduced glare, and permanent functionality because it utilizes a novel system of polymeric capsule and filling material to enhance the optical performance of the eye and establish normal visual experience.

Further, the presbyopia correcting IOLs of the present invention are advantageous because this novel material is softer than the material currently used in optics of presbyopia correcting IOLs and could allow for improvements in the optics of these premium IOLs. This material might allow for the molding of unique diffractive surfaces on the IOL. In some embodiments, the IOL may be molded using a TEFLON™ mold as described herein. Thus, this material could be used to make better presbyopia correcting IOLs. Alternatively, this novel material could be used in an IOL that may or may not be an A-IOL or a presbyopia correcting IOL, but is a custom-made IOL.

Since custom-made IOLs do not exist, there are certain ocular problems that cannot be corrected by current IOLs, such as irregular astigmatism. Advanced scans of the front of the eye, such as corneal topography, provide a map of the shape and curvature of the cornea. Using corneal topography, aberrometry (wavefront technology), and other imaging and diagnostic tools, one could create a computer model of the ideal shape of an IOL or an accommodating IOL for each patient. Using the computer model, a mold could be made, and the Generation 2 material could be molded inside of it to a create custom-made IOL to correct for each specific eye's needs. In addition to correcting for corneal aberrations, a custom-made IOL or accommodating IOL could be molded to fit perfectly into each patient's capsular bag. Ultrasound or MRI imaging of each patient's lens could be used to determine details such as lens equatorial diameter, volume, and surface area. These variables could also be plugged into a computer model of the eye to help determine the ideal shape of an IOL for each patient. Thus, this novel material could be used to create custom-made IOLs.

The following terms may have meanings ascribed to them below, unless specified otherwise. As used herein, the terms “comprising,” “to comprise” and the like do not exclude the presence of further elements or steps in addition to those listed in a claim. Similarly, the terms “a,” “an” or “the” before an element or feature does not exclude the presence of a plurality of these elements or features, unless the context clearly dictates otherwise.

Throughout this application, the term “about” is used to indicate that a value includes the inherent variation of error for the device, or the method being employed to determine the value, or the variation that exists among the samples being measured. Unless otherwise stated or otherwise evident from the context, the term “about” means within 10% (i.e., within 10%, 9%, 8%, 7%, 6%, 5%, 4%, 3%, 2%, 1%, or less) above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents. Unless otherwise clear from context, all numerical values provided herein in the specification and the claim can be modified by the term “about.”

It should also be understood that the ranges provided herein are a shorthand for all the values within the range and, further, that the individual range values presented herein can be combined to form additional non-disclosed ranges. For example, a range of 1 to 50 is understood to include not only 1 and 50, but any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

A polymer “comprises” or is “derived from” a stated monomer if that monomer is incorporated into the polymer. Thus, the incorporated monomer that the polymer comprises is not the same as the monomer prior to incorporation into a polymer, in that at the very least, certain terminal groups are incorporated into the polymer backbone. A polymer is said to comprise a specific type of linkage if that linkage is present in the polymer.

As used herein, the term “homopolymer” refers to a polymer derived from a single monomeric species. And as follows, unless otherwise indicated, the term “copolymer” refers to a polymer derived from two, three or more monomeric species and includes alternating copolymers, periodic copolymers, random copolymers, statistical copolymers and block copolymers. Unless otherwise indicated, the term “block copolymer” comprises two or more homopolymer or copolymer subunits linked by covalent bonds.

As used herein, the term “residue(s)” is used to refer generally to the part of a monomer or other chemical unit that has been incorporated into a polymer or large molecule. By extension, the terms “residue of the chain transfer agent” and the “chain transfer agent residue” are used interchangeably to refer to the parts of the chain transfer agent that have been incorporated into the bottlebrush polymers. Conversely, a polymer “comprises” or is “derived from” a stated monomer if that monomer is incorporated into the polymer. Thus, the incorporated monomer that the polymer comprises is not the same as the monomer prior to incorporation into a polymer, in that at the very least, certain terminal groups are incorporated into the polymer backbone. A polymer is said to comprise a specific type of linkage if that linkage is present in the polymer.

As used herein the terms “functional group” and “functional moiety” are used interchangeably to refer a chemically active species, or a group containing a chemically active species. As used herein, the term “functionalized” refers to a polymer or other substance that includes, or has been modified to include, a functional group, and the broader term “functionalization” refers to a process, method and/or reaction whereby a functional group is added to a polymer or other substance.

The term “ultra-violet light” is used herein to refer to light having a wavelength of from about 10 nm to about 400 nm. Similarly, the term “ultra-violet light blocking” as applied to a polymer or other material, refers broadly to the ability that polymer or material to block or reduce transmission of ultra-violet light or to a polymer or other material having that ability.

All publications, patent applications, patents, and other references mentioned herein are expressly incorporated by reference in their entirety, which means that they should be read and considered by the reader as part of this text. That the document, reference, patent application, or patent cited in this text is not repeated in this text is merely for reasons of conciseness. In the case of conflict, the present disclosure, including definitions, will control. All technical and scientific terms used herein have the same meaning, unless otherwise indicated.

Further, any compositions or methods provided herein can be combined with one or more of any of the other compositions and methods provided herein. The fact that given features, elements or components are cited in different dependent claims does not exclude that at least some of these features, elements or components maybe used in combination together.

In a first aspect, the present invention is directed to a bottlebrush polymer or copolymer for use as a custom-molded material in ocular implants comprising a homopolymer or copolymer of one or more tri-arm bottlebrush polymers. In one or more embodiment, the tri-arm star bottlebrush polymer of the present invention will comprise the residue of a trifunctional reversible addition fragmentation chain-transfer (RAFT) agent and three methacrylate or acrylate polymer chains extending therefrom. In these embodiments, each of said three methacrylate polymer chains comprising the polymerized residues of at least one methacrylate macromonomer.

In various embodiments, the trifunctional reversible addition fragmentation chain-transfer (RAFT) agent will have three sites capable of reversible addition fragmentation chain-transfer (RAFT) polymerization. In one or more embodiments, trifunctional reversible addition fragmentation chain-transfer (RAFT) agent will be a three arm, branched molecule with each of the branches having a RAFT capable, thiol containing, end group. In some embodiments, the trifunctional RAFT agent will have three thiol, dithiobenzoate, or trithiocarbonate end groups. In one or more embodiments, the trifunctional RAFT agent is 1,1,1-tris [(dodecylthiocarbonothioylthio)-2-methylpropionate]ethane (Tris (DDMAT)).

In one or more embodiment, the trifunctional RAFT agent will have the formula:

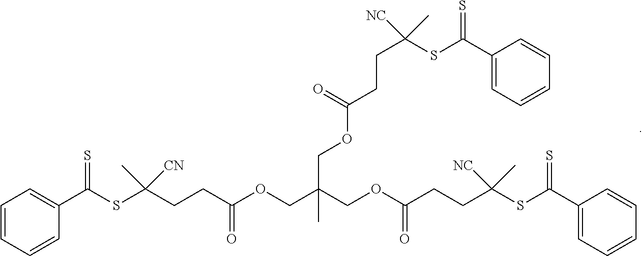 [SMILES: CC(COC(=O)CCC(C)(C#N)SC(=S)c1ccccc1)(COC(=O)CCC(C)(C#N)SC(=S)c1ccccc1)COC(=O)CCC(C)(C#N)SC(=S)c1ccccc1]

Each of the three methacrylate or acrylate polymer chains will comprise residues of methacrylate macromolecule monomer selected from the group consisting of monomethacryloxypropyl terminated polydimethylsiloxane, asymmetric (PDMS-MA) and oligo (ethylene glycol) methacrylate (OEGMA), but is preferably PDMS-MA. In some embodiments, the methacrylate macromonomers have the formula:

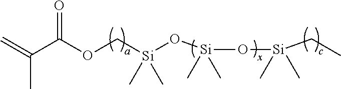

where a is an integer from about 1 to about 6; c is an integer from 1 to 5; and x is an integer from about 2 to about 20. In some embodiments, a is an integer from about 1 to about 5, in other embodiments, from about 1 to about 3, in other embodiments, from about 2 to about 6, in other embodiments, from about 3 to about 6, and in other embodiments, from about 4 to about 6. In one or more embodiment, a is 3. In some embodiments, c is an integer from about 1 to about 4, in other embodiments, from about 1 to about 3, in other embodiments, from about 2 to about 5, and in other embodiments, from about 3 to about 5. In some embodiments, x is an integer from about 2 to about 18, in other embodiments, from about 2 to about 14, in other embodiments, from about 2 to about 10, in other embodiments, from about 2 to about 6, in other embodiments, from about 4 to about 20, in other embodiments, from about 8 to about 20, in other embodiments, from about 12 to about 20, in other embodiments, from about 16 to about 20, and in other embodiments, from about 18 to about 20. In some embodiments x is an integer from about 5 to about 10. In one or more embodiments, x is 6 or 8.

In some of these embodiments, the methacrylate macromonomers have the formula:

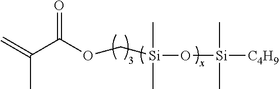

where x is an integer from about 5 to about 10 and will have mass average molecular weight from about 600 kDa to about 800 kDa.

In some other embodiments, the methacrylate macromonomers have the formula:

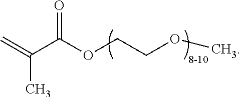

In various embodiments, the methacrylate macromonomers and trifunctional RAFT agents described above are reacted using RAFT polymerization techniques as described in detail below, to form a tri-arm star bottlebrush polymer. In some of these embodiments, the tri-arm star bottlebrush polymer of the present invention will have the formula:

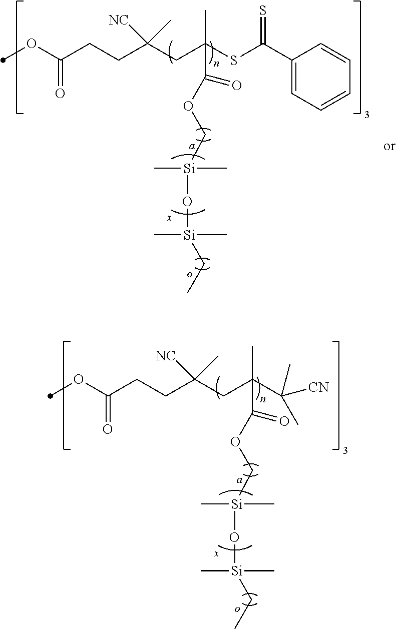

where a is an integer from about 1 to about 6; c is an integer from 1 to 5; x is an integer from about 2 to about 20 and n is an integer from about 10 to about 80. In some embodiments, n is an integer from about 10 to about 70, in other embodiments, from about 10 to about 50, in other embodiments, from about 10 to about 30, in other embodiments, from about 20 to about 80, in other embodiments, from about 20 to about 80, in other embodiments, from about 30 to about 80, in other embodiments, from about 40 to about 80, in other embodiments, from about 60 to about 80, and in other embodiments, from about 60 to about 80. In various embodiments, a, c, and x are as set forth above for the macromonomers.

In some of these embodiments, the tri-arm star bottlebrush polymer of the present invention will have the formula:

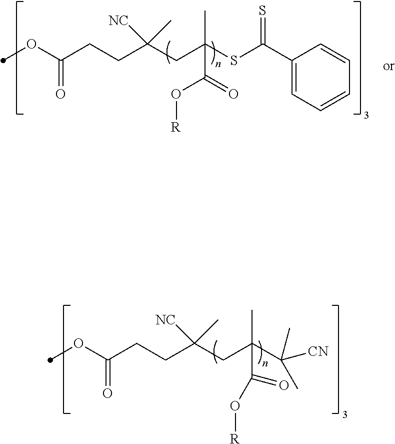

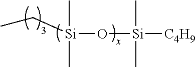

wherein R has the formula where x is an integer from about 5 to about 10; and n is an integer from about 10 to about 80. In various embodiments, x and n may be as set forth above.

Further, to adjust the intrinsic viscosity, tune the mechanical properties, or add additional properties, the three methacrylate or acrylate polymer chains may include residues of other methacrylates and/or acrylates. Suitable methacrylate or acrylate monomers may include, without limitation, 2,2,2-trifluoroethyl methacrylate (TFEMA), 3,3,4,4,5,5,6,6,7,7,8,8,9,9,10,10,10-heptadecafluorodecyl methacrylate (HDFDMA), benzyl methacrylate (BzMA), 2-[3-(2H-Benzotriazol-2-yl)-4-hydroxyphenyl]ethyl methacrylate (BzTAzMA), ethyleneglycol phenylether methacrylate (EGPhEMA), hydroxyethyl methacrylate (HEMA), 2,2,2-trifluoroethyl acrylate (TFEA), 3,3,4,4,5,5,6,6,7,7,8,8,9,9,10,10,10-heptadecafluorodecyl acrylate (HDFDA), benzyl acrylate (BzA), 2-[3-(2H-Benzotriazol-2-yl)-4-hydroxyphenyl]ethyl acrylate (BzTAzA), ethyleneglycol phenylether acrylate (EGPhEA), hydroxyethyl acrylate (HEA), and combinations thereof, in some embodiments, fluorinated methacrylic monomers are copolymerized with PDMS-MA to decrease the nᵣ and benzylic monomers are copolymerized with OEGMA to reach the upper limit of nᵣ. In these embodiments, the final nᵣ range is 1.40-1.48. Additionally, it is possible to incorporate a UV-absorbing reagent into these methacrylate or acrylate polymer chains to filter out UV light.

In addition, the three methacrylate or acrylate polymer chains tri-arm star bottlebrush polymer of the present invention may also contain residues of one or more hydroxy-functionalized methacrylate chain extenders, such as 2-hydroxyethyl methylacrylate (HEMA) molecules. In some embodiments, the tri-arm star bottlebrush polymer will have a formula selected from:

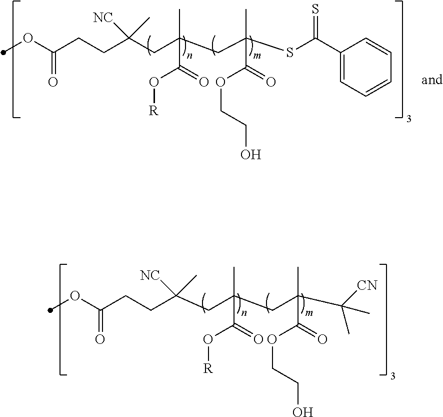

wherein R has the formula

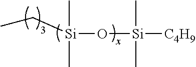

where x is an integer from about 5 to about 10; n is a mole percent from about 80% to about 99%; and m is a mole percent from about 1% to about 20%. In some embodiments, n is from about 80 mol. % to about 98 mol. %, in other embodiments, from about 80 mol. % to about 95 mol. %, in other embodiments, from about 80 mol % to about 92 mol. %, in other embodiments, from about 80 mol. % to about 90 mol. %, in other embodiments, from about 80 mol. % to about 85 mol. %, in other embodiments, from about 85 mol. % to about 99 mol. %, in other embodiments, from about 88 mol % to about 99 mol. %, in other embodiments, from about 93 mol. % to about 99 mol. %, and in other embodiments, from about 96 mol % to about 99 mol. %. In some of these embodiments, n is a mole percent from about 90% to about 99%. In other embodiments, n is a mole percent from about 95% to about 99%. Here, as well as elsewhere in the specification and claims, individual range values can be combined to form additional non-disclosed ranges.

In various embodiments, m is a mole percent from about 1% to about 20%, in other embodiments, from about 1 mol. % to about 15 mol. %, in other embodiments, from about 1 mol. % to about 12 mol. %, in other embodiments, from about 1 mol. % to about 9 mol. %, in other embodiments, from about 1 mol % to about 6 mol. %, in other embodiments, from about 1 mol. % to about 3 mol. %, in other embodiments, from about 3 mol. % to about 20 mol. %, in other embodiments, from about 5 mol. % to about 20 mol. %, and in other embodiments, from about 10 mol. % to about 20 mol %. In some of these embodiments, m is from about 1 mol. % to about 10 mol. %. In other embodiments, m is from about 1 mol. % to about 5 mol. %. Here, as well as elsewhere in the specification and claims, individual range values can be combined to form additional non-disclosed ranges.

In some of these embodiments, the PDMS-MA and HEMA are added in sequence as set forth below. In these embodiments, the three methacrylate polymer chains will each comprise an A: B block co polymer having a PDMS-MA A block and a HEMA B block.

As set forth above, two or more hydroxy-functionalized methacrylate chain extenders can be modified at their terminal hydroxy groups to have alkene functional groups. In some embodiments, the tri-arm star bottlebrush may have a formula selected from:

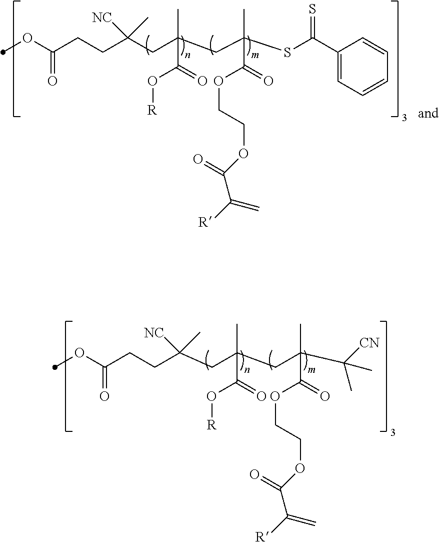

wherein R has the formula

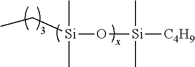

where x is an integer from about 5 to about 10; R′ is H or CH₃; n is a mole percent from about 80% to about 99%; and m is a mole percent from about 1% to about 20%. In various embodiments, n and m may be as set forth above.

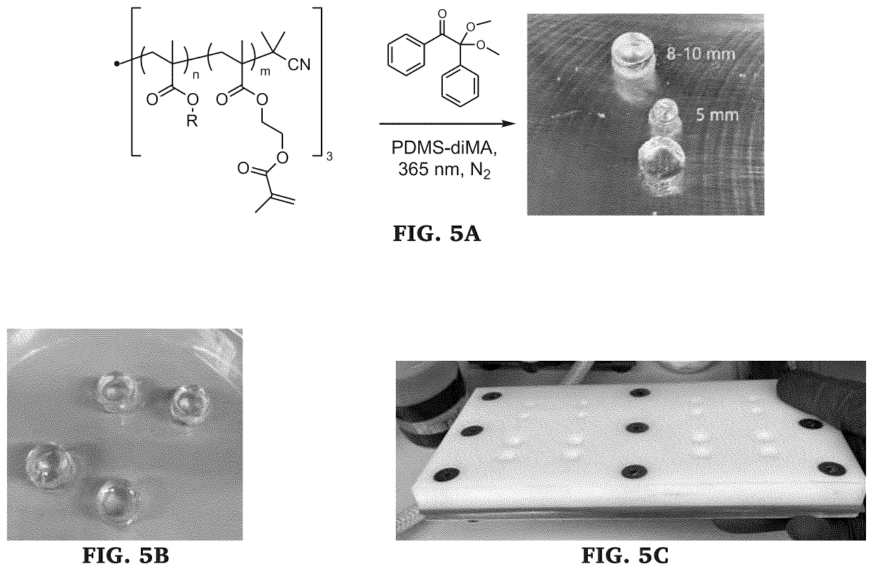

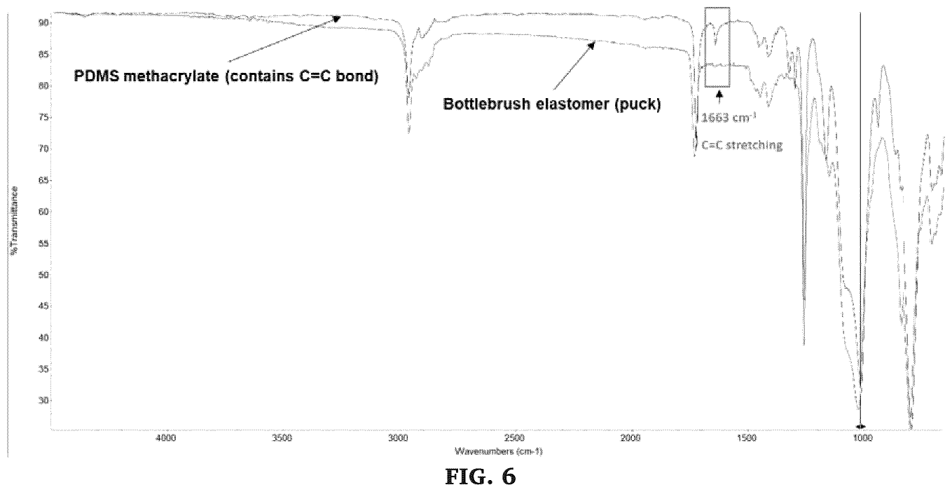

As will be appreciated by those of skill in the art, during the RAFT polymerization reaction, the methacrylate macromonomers will polymerize at a location at or near the center of the RAFT agent and leaving the terminal thiol groups on the trifunctional RAFT agent in place. In embodiments where Tris (DDMAT) is used as the trifunctional RAFT agent, for example, the three methacrylate chains formed during RAFT polymerization will all have terminal trithiolcarbonate groups. While the bottlebrush polymers of the present invention are all transparent after formation by RAFT polymerization, they are often tinted and not fully optically clear. Advantageously, however, it has been found that removing the sulfur containing end group (a residue of the RAFT agent) produces a polymer that is optically clear. So, in embodiments where the bottlebrush polymers of the present invention required to be optically clear, these thiol containing end groups may be removed by the addition of an excess of thermally or chemically activated radical generating compound, such as 2,2′-azobis(2-methylpropionitrile) (AIBN). The resulting polymers are optically clear, as shown in FIGS. 5A and 5B.

In various embodiments, the tri-arm star bottlebrush polymer will have a formula selected from:

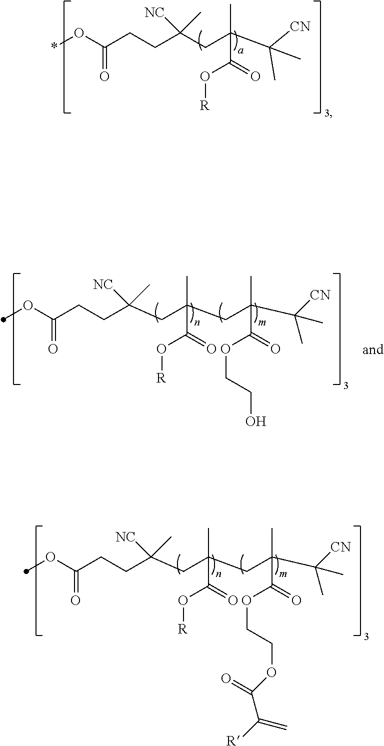

wherein R has the formula

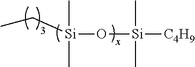

where x is an integer from about 5 to about 10; R′ is H or CH₃; a is an integer from about 10 to about 80; n is a mole percent from about 80% to about 99%; and m is a mole percent from about 1% to about 20%. In various embodiments, n and m may be as set forth above. In these embodiments, the tri-arm star bottlebrush polymers of the present invention are optically clear.

In one or more embodiments, the tri-arm star bottlebrush polymer has a refractive index of from about 1.40 to about 1.49, preferably from about 1.42 to about 1.48, and more preferably from about 1.43 to about 1.46 at 37° C. In various embodiments, the tri-arm star bottlebrush polymer of the present invention will have a degree of polymerization for each arm between about 10 and about 80. In some embodiments, the tri-arm star bottlebrush polymer of the present invention will have a degree of polymerization for each arm of from about 2 to about 20 and n is an integer from about 10 to about 80. In some embodiments, n is an integer from about 10 to about 70, in other embodiments, from about 10 to about 50, in other embodiments, from about 10 to about 30, in other embodiments, from about 20 to about 80, in other embodiments, from about 20 to about 80, in other embodiments, from about 30 to about 80, in other embodiments, from about 40 to about 80, in other embodiments, from about 60 to about 80, and in other embodiments, from about 60 to about 80. Here, as well as elsewhere in the specification and claims, individual range values can be combined to form additional non-disclosed ranges.

In a second aspect, the present invention is directed to a photocurable tri-arm star bottlebrush polymer resin comprising the tri-arm star bottlebrush polymer described above, a bis-methacryl terminated polydimethylsiloxane crosslinker and a suitable photoinitiator. In some embodiments, the bismethacryl terminated polydimethylsiloxane crosslinker is dimethacryloxypropyl terminated polydimethylsiloxane (PDMS-diMA), or a combination thereof. In one or more embodiment, the bis-methacryl terminated polydimethylsiloxane crosslinker will have the formula:

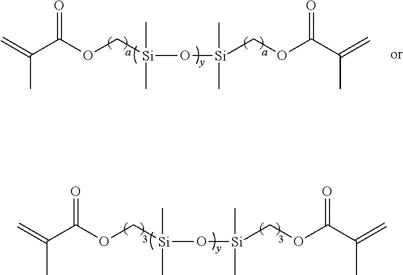

where a is an integer from about 1 to about 6 and y is an integer from about 2 to about 30. In some embodiments, x is an integer from about 2 to about 25, in other embodiments, from about 2 to about 20, in other embodiments, from about 2 to about 15, in other embodiments, from about 2 to about 10, in other embodiments, from about 5 to about 30, in other embodiments, from about 10 to about 30, in other embodiments, from about 15 to about 30, in other embodiments, from about 20 to about 30, and in other embodiments, from about 25 to about 30. Here, as well as elsewhere in the specification and claims, individual range values can be combined to form additional non-disclosed ranges.

In some of these embodiments, the photocurable tri-arm star bottlebrush polymer resin will comprise from about 2% to about 98% PDMS-diMA by volume. In some embodiments, the photocurable tri-arm star bottlebrush polymer resin will comprise from about 2% to about 95%, in other embodiments, from about 2% to about 80%, in other embodiments, from about 2% to about 60%, in other embodiments, from about 2% to about 40%, in other embodiments, from about 2% to about 20%, in other embodiments, from about 10% to about 98%, in other embodiments, from about 30% to about 98%, in other embodiments, from about 50% to about 98%, and in other embodiments, from about 70% to about 98% PDMS-diMA by volume.

The photoinitiator is not particularly limited and any photoinitiator capable of generating radicles upon exposure to ultraviolet light may be used provided that it is miscible with the monomer. Suitable photoinitiators may include, without limitation, 2,2-dimethoxy-1,2-diphenylethanone. In some embodiments, the photoinitiator is 2,2-dimethoxy-1,2-diphenylethanone. In one or more embodiment, the photocurable tri-arm star bottlebrush polymer resin is optically clear.

In some embodiments, the photocurable tri-arm star bottlebrush polymer resin will comprise from about 2 vol. % to about 98 vol. % tri-arm star bottlebrush polymer, from about 2 vol. % to about 98 vol. % PDMS-diMA, and an operable amount of 2,2-dimethoxy-1,2-diphenylethanone. In some other embodiments, the photocurable tri-arm star bottlebrush polymer resin will comprise from about 95 vol. % to about 98 vol. % tri-arm star bottlebrush polymer, from about 2 vol. % to about 4 vol. % PDMS-diMA, and an operable amount of 2,2-dimethoxy-1,2-diphenylethanone. As used herein, the term “operable amount” as applied to the photoinitiator, refers to a quantity of photoinitiator sufficient to produce enough radicals to drive the RAFT polymerization reaction to completion. One of ordinary skill in the art will be able to determine an operable amount of photoinitiator without undue experimentation.

In a third aspect, the present invention is directed to a bottlebrush hydrogel network comprising a photocured tri-arm star bottlebrush polymer resin described above, for use in artificial intraocular lenses comprising the photocurable tri-arm star bottlebrush polymer resin described above. In some embodiments, the photocured tri-arm star bottlebrush polymer resin has a Young's modulus of from about 0.005 MPa to about 0.05 MPa. In some embodiments, the photocured tri-arm star bottlebrush polymer resin has a Young's modulus of from about 0.005 MPa to about 0.04 MPa, in other embodiments, from about 0.005 MPa to about 0.03 MPa, in other embodiments, from about 0.005 MPa to about 0.03 MPa, in other embodiments, from about 0.005 MPa to about 0.02 MPa, in other embodiments, from about 0.005 MPa to about 0.01 MPa, in other embodiments, from about 0.01 MPa to about 0.05 MPa, in other embodiments, from about 0.02 MPa to about 0.05 MPa, in other embodiments, from about 0.03 MPa to about 0.05 MPa, and in other embodiments, from about 0.02 MPa to about 0.05 MPa. Here, as well as elsewhere in the specification and claims, individual range values can be combined to form additional non-disclosed ranges.

In various embodiments, the photocured tri-arm star bottlebrush polymer resin has an ultimate compressive strength (UCS) of from about 0.002 MPa to about 0.5 MPa after curing with ultraviolet light. In some embodiments, the photocured tri-arm star bottlebrush polymer resin has an ultimate compressive strength (UCS) of from about 0.002 MPa to about 0.4 MPa, in other embodiments, from about 0.002 MPa to about 0.3 MPa, in other embodiments, from about 0.002 MPa to about 0.2 MPa, in other embodiments, from about 0.002 MPa to about 0.1 MPa, in other embodiments, from about 0.002 MPa to about 0.01 MPa, in other embodiments, from about 0.005 MPa to about 0.4 MPa, in other embodiments, from about 0.01 MPa to about 0.4 MPa, in other embodiments, from about 0.1 MPa to about 0.4 MPa, and in other embodiments, from about 0.2 MPa to about 0.4 MPa, after curing with ultraviolet light. Here, as well as elsewhere in the specification and claims, individual range values can be combined to form additional non-disclosed ranges. In some embodiments, the photocured tri-arm star bottlebrush polymer resin is optically clear.

In a fourth aspect, the present invention is directed to artificial intraocular lenses (IOLs) for use in treating cataracts comprising a custom-molded intraocular lens comprising one or more of the bottlebrush polymers and copolymers described above. In various embodiments, the artificial intraocular lenses (IOLs) In these embodiments, the bottlebrush polymers and copolymers used will have a refractive index (nᵣ) of from about 1,40 to about 1.49, preferably from about 1.42 to about 1.48, and more preferably from about 1.43 to about 1.46 at 37° C. In some embodiments, the artificial intraocular lens of the present invention will have a Young's modulus of from about 0.005 MPa to about 0.05 MPa, as set forth above. In some embodiments, the artificial intraocular lens of the present invention will have an ultimate compressive strength (UCS) of from about 0.002 MPa to about 0.5 MPa, as set forth above. In various embodiments, the artificial intraocular lens of the present invention is optically clear.

As set forth above, the natural lens loses its elasticity over time, growing thicker and less flexible leading to presbyopia. With age, the lens become thicker and opaquer, leading to blurred vision and cataracts. The artificial lens according to the present invention is implanted in the eye of a patient to replace a lens that has become thicker, less flexible and opaquer with age. This A-IOL should have a refractive index (nᵣ) between 1.40-1.49 and complex viscosity to allow it to be deformed by the muscles of the eye to allow the eye to focus. The present disclosure provides a solution for presbyopia and cataracts with an accommodating intraocular lens that can change shape in response to the muscles of accommodation and obviate the need for eyeglasses and contact lenses by providing clear vision over a range of distances.

In some embodiments, the artificial lens will be an accommodating intraocular lens (A-IOL). In some embodiments, the intraocular lens will be a pseudoaccommodating presbyopia correcting IOL. In some embodiments, the intraocular lens will not be accommodating. In some embodiments, the artificial lens will be a custom-molded lens, made specifically for one patient's eye. In some embodiments, the IOL may be an intraocular lens as described in U.S. Pat. No. 10,278,810, US Patent Application Publication 2019/0321163 A1, or International Application Number PCT/US20/52316, the disclosures of which are incorporated herein by reference in their entirety.

As will be understood by those skilled in the art, the lens of the eye is acted upon by the muscles of accommodation which change the shape of the lens to allow the eye to focus over a range of distances. People with young healthy eyes can focus on objects at near through a process called accommodation. During accommodation, there is an increase in the optical power of the eye's crystalline lens due to an increase in lens axial thickness, an increase in curvature of the lens anterior and posterior surfaces, and a decrease in lens diameter.

According to the Helmholtz theory of accommodation, when an eye is focused at distance, the circular ciliary muscle is relaxed and the zonules pull on the lens, flattening it. When the focuses on a near object, the ciliary muscle contracts, and the lens zonules slacken. With the decreased zonular tension, the lens becomes thicker and more convex. This rounder lens leads to an increase in the dioptric power of the eye, allowing for near vision. In the Helmholtz theory, the zonules are relaxed during accommodation and are under tension when accommodation ends. (Glasser 2006)

In a fifth aspect, the present invention includes a method for making a photocured tri-arm star bottlebrush polymer resin. In these embodiments, a tri-arm star bottlebrush polymer having desired properties is first combined with PDMS-diMA, and a photoinitiator to form an uncured tri-arm star bottlebrush polymer resin, as set forth above. In some of these embodiments, the uncured tri-arm star bottlebrush polymer resin comprises from about 2% to about 98% by volume PDMS-diMA and the photoinitiator is 2,2-dimethoxy-1,2-diphenylethanone. In some embodiments, the uncured tri-arm star bottlebrush polymer resin comprises from about 2% to about 95%, in other embodiments, from about 2% to about 85%, in other embodiments, from about 2% to about 75%, in other embodiments, from about 2% to about 65%, in other embodiments, from about 2% to about 55%, in other embodiments, from about 2% to about 45%, in other embodiments, from about 2% to about 35%, in other embodiments, from about 2% to about 25%, in other embodiments, from about 2% to about 15%, in other embodiments, from about 5% to about 98%, in other embodiments, from about 10% to about 98%, in other embodiments, from about 20% to about 98%, in other embodiments, from about 30% to about 98%, in other embodiments, from about 40% to about 98%, in other embodiments, from about 50% to about 98%, in other embodiments, from about 60% to about 98%, in other embodiments, from about 70% to about 98%, in other embodiments, from about 80% to about 98% and in other embodiments, from about 90% to about 98% PDMS-diMA by volume. Here, as well as elsewhere in the specification and claims, individual range values can be combined to form additional non-disclosed ranges.

The uncured tri-arm star bottlebrush polymer resin is then cured by exposing it to ultraviolet light to produce a photocured tri-arm star bottlebrush polymer resin. In one or more embodiments, the ultraviolet light used to cure the uncured tri-arm star bottlebrush polymer resin has a wavelength of from about 300 nm to about 600 nm.

In various embodiments, the photocured tri-arm star bottlebrush polymer resin has a Young's modulus of from about 0.005 MPa to about 0.05 MPa and an ultimate compressive strength (UCS) of from about 0.002 MPa to about 0.5 MPa, as set forth above. In various embodiments, photocured tri-arm star bottlebrush polymer resin produced is also optically clear.

In a sixth aspect, the present invention includes a method of making an artificial intraocular lens comprising the tri-arm star bottlebrush polymers described above. In these embodiments, a tri-arm star bottlebrush polymers having the desired refractive index and physical properties is selected and combined with PDMS-diMA, and a photoinitiator to form an uncured tri-arm star bottlebrush polymer resin, as set forth above. A mold shaped to hold an artificial intraocular lens of a desired size and shape is prepared and filled with an appropriate amount of the uncured tri-arm star bottlebrush polymer resin. The mold may formed be by any suitable means known in the art. The mold is preferably formed of, or coated with, a non-stick material to allow easy removal of the newly formed IOL from the custom-made mold. In some embodiments, the mold is made from or coated with TEFLON™, as shown in FIG. 5C. Finally, exposing the uncured tri-arm star bottlebrush polymer resin to ultraviolet light to produce an artificial intraocular lens comprising photocured tri-arm star bottlebrush polymer resin and having the desired size, shape, refractive index and physical properties. In some embodiments, the uncured tri-arm star bottlebrush polymer resin to ultraviolet light having a wavelength of from about 300 nm to about 600 nm.

In yet another aspect, the present invention is directed to a method of making a non-accommodating presbyopia correcting IOL comprising the tri-arm star bottlebrush polymer described above that is formed into an optic of an IOL. The anterior or posterior surface of this presbyopia correcting IOL could possess concentric diffractive surfaces to allow for a good range of vision.

In yet another aspect, the present invention is directed to a method of making an artificial accommodative IOL comprising the tri-arm star bottlebrush polymer described above that is tailored for the needs of a particular patient. In these embodiments, an ideal shape or at least a desired shape for an IOL or an accommodating IOL is determined by measurement and analysis of a patients existing intraocular lens and other eye anatomy. As set forth above, advanced scans of the front of the eye, such as corneal topography, provide a map of the shape and curvature of the cornea. In addition to correcting for corneal aberrations, a custom-made accommodating IOL could be molded to fit perfectly into each patient's capsular bag. Ultrasound or MRI imaging of each patient's lens could be used to determine details such as lens equatorial diameter, volume, and surface area. As will be appreciated by those of ordinary skill in the art, these variables may also be plugged into a computer model of the eye to help determine the ideal shape of an IOL or A IOL for each patient. Using corneal topography, aberrometry (wavefront technology), optical coherence tomography (OCT), and other imaging and diagnostic tools, a computer model of the ideal shape of an IOL or accommodative IOL for each patient is generated. One of ordinary skill in the art would be able to generate such a computer model without undue experimentation using currently available computer imaging technologies.

Using the computer model, a mold having the required shape is then generated by any suitable means known in the art. The mold is preferably formed of or coated with a non-stick material to allow easy removal of the newly formed IOL from the custom-made mold. In some embodiments, the mold is made from or coated with TEFLON™, as shown in FIG. 5C.

Next, a tri-arm star bottlebrush polymer having the required refractive index and other desired properties upon curing is selected and combined with PDMS-diMA, and a photoinitiator to form an uncured tri-arm star bottlebrush polymer resin as set forth above. An amount of the uncured tri-arm star bottlebrush polymer resin approximately equal to the volume of the desired artificial accommodative IOL is then added to fill the mold and the mold is exposed to ultraviolet light as described above to produce an artificial intraocular lens having an ideal shape for said patient and comprising photocured tri-arm star bottlebrush polymer resin.

### EXAMPLES

The following examples are offered to more fully illustrate the invention but are not to be construed as limiting the scope thereof. Further, while some of examples may include conclusions about the way the invention may function, the inventors do not intend to be bound by those conclusions but put them forth only as possible explanations. Moreover, unless noted by use of past tense, presentation of an example does not imply that an experiment or procedure was, or was not, conducted, or that results were, or were not actually obtained. Efforts have been made to ensure accuracy with respect to numbers used (e.g., amounts, temperature), but some experimental errors and deviations may be present. Unless indicated otherwise, parts are parts by weight, molecular weight is weight average molecular weight, temperature is in degrees Centigrade, and pressure is at or near atmospheric.

### Materials

Unless otherwise noted, solvents were received from Fisher Scientific as ACS grade and used without further purification. Anhydrous toluene for reversible addition-fragmentation chain-transfer (RAFT) polymerization and anhydrous THF for end-group removal (EGR) were used from the Inert PureSolv Solvent Purification System. 1,1,1-tris [(dodecylthiocarbonothioylthio)-2-methylpropionate] ethane (Tris (DDMAT) or CTA3, 98%, Sigma-Aldrich), 2,2-dimethoxy-2-phenylacetophenone (DMPA, 99%, Sigma-Aldrich), methacryloxypropyl terminated polydimethylsiloxane (PDMS-diMA, DMS-R11, MW=900-1,200 g/mol, Gelest), methacryloyl chloride (97%, Sigma-Aldrich), triethylamine (Et₃N or TEA, 99.5%, Sigma-Aldrich), and chloroform-d (CDCl₃, 99.8 atom % D, contains 0.03% v/v TMS, Sigma-Aldrich) were used as received. 2,2′-Azobis(2-methylpropionitrile) (AIBN, 98%, Sigma-Aldrich) was recrystallized from MeOH. Monomethacryloxypropyl terminated polydimethylsiloxane-asymmetric (PDMS-MA, MCR-M07, MW=600-800 g/mol, Gelest) and 2-hydroxyethyl methylacrylate (HEMA, 99%, Sigma-Aldrich) were freshly purified by passing through a short column of basic alumina before use.

### Instrumentation

NMR spectroscopy analysis of the samples were collected using a Bruker Advance Neo 500 MHz multinuclear NMR spectrometer. Chemical shifts are reported in ppm (δ) and referenced to the residual CHCl₃ proton resonance at 7.26 ppm in CDCl₃. Size exclusion chromatography (SEC) was performed using an HLC-8420GPC, EcoSEC Elite Gel Permeation Chromatography (GPC) System (Tosoh Bioscience, LLC), equipped with UV, RI, and LenS3 multi-angle light scattering (MALS) detectors, TSKgel GMHHR-M mixed bed sample column (7.8 mm ID×30 cm, 5 μm). The number average molecular mass (Ma), weight average molecular mass (M_{w}), and molecular mass distribution (Ð_{M}) for each sample (A concentration of 5-10 mg/mL sample was prepared and 15 μL injection volume was used) were calculated using poly(styrene) cocktails for UV and RI detectors and a narrow poly(styrene) standard (Tosoh TSKgel standard, M_{w}=37,900 g/mol, M_{w}/Mₙ=1.01) for MALS detector and using light scattering distribution and peak-average analyses by low-angle light scattering (LALS) and right-angle light scattering (RALS) in THE SEC system flowing 0.35 mL/min at 40° C.

The viscosity of the neat polymer melts was measured using a TA Instruments Discovery Hybrid Rheometer 3 (DHR 3). Each polymer melt was placed between parallel plates (25 mm diameter) using a 200 μm gap, and data was collected via an angular frequency sweep ranging from 0.1 rad/s to 500 rad/s at 10% strain at 25, 37, 45, and 50° C. Refractive index measurements were performed with neat polymer melts using a Bellingham & Stanley RFM 340 with a chiller at 25 and 37° C. UV-Vis spectroscopy analysis was performed in solution using Shimadzu UV-3600i UV-Vis-NIR spectrophotometer and a quartz cuvette with 10 mm path length.

### Example 1

### Synthesis of Three Arm Star Bottlebrush (BB) Polymers

Three-arm star bottlebrush polymers were synthesized by RAFT polymerization of PDMS-MA using tri-functional chain transfer agent as shown in Scheme 1, below.

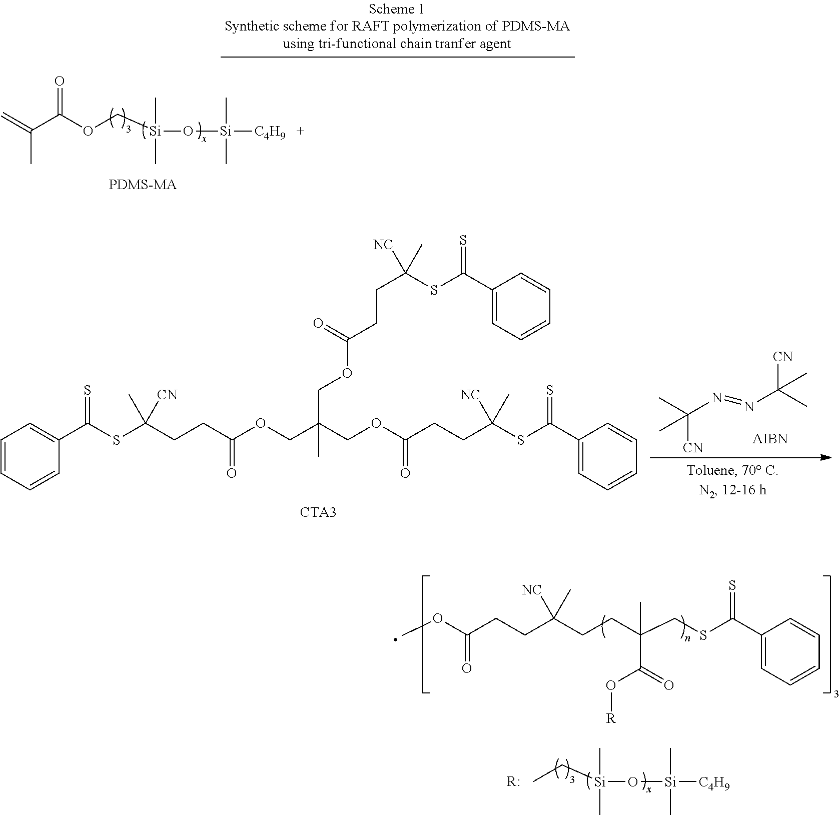

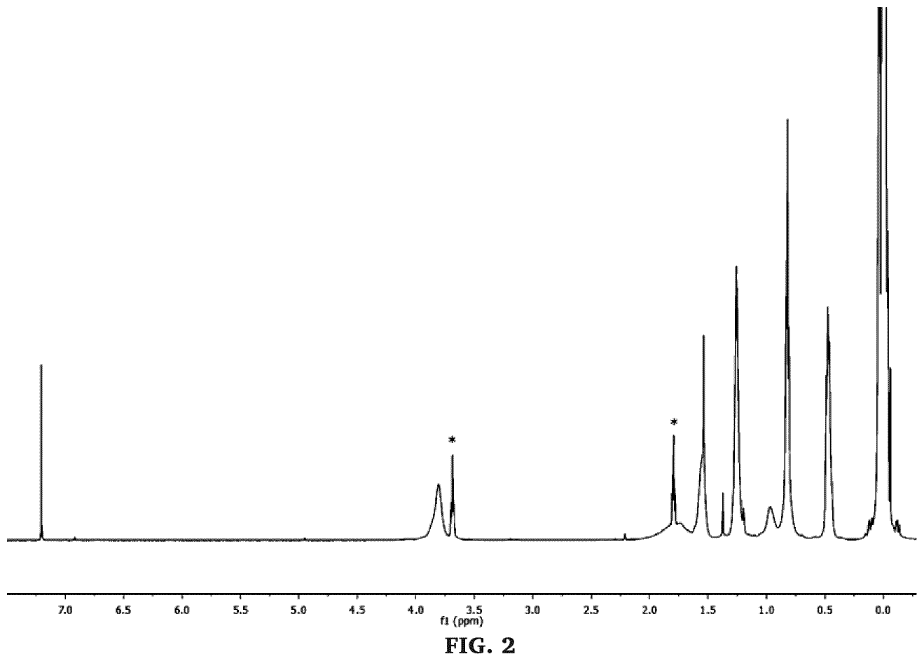

A typical RAFT polymerization was conducted as follows: To a Schlenk flask equipped with a Teflon coated micro stir bar, purified PDMS-MA macromonomer (M, 10.0 mL, 13.71 mmol, ca. 100 equiv.), tri-functional RAFT agent (CTA3, 159.1 mg, 0.1371 mmol, 1 equiv.), AIBN initiator (I, 11.46 mg, 0.0686 mmol, 0.5 equiv.), and anhydrous toluene (5.0 mL) were added and sealed with a septum. The mixture was sparged with N₂ for 20-30 minutes. Then, the flask was placed in a pre-heated oil bath at 70° C. The polymerization was run for 12-16 hours. The polymerization was quenched by opening the flask to air and adding 10-15 mL of MeOH directly to the flask. The resulting mixture was vortexed, sonicated, and placed in an ice bath for a couple of minutes. Then, the top liquid layer was decanted, and this purification step was repeated 2 to 4 more times. The final polymer was dissolved in THE, the solution was passed through a 1 μm PTFE filter, all the volatiles were removed under reduced pressure using a rotovap (typically 85-90 mbar, 35-40° C.), and the resulting viscous liquid polymer was dried at high vacuum at room temperature overnight. Yellow colored, transparent, viscous liquid polymer melt was obtained (>95% monomer conversion, 7.8 g isolated yield). M_{w,SEC}=71,450 g/mol, Ð_{M}=1.37. Refractive index (n)=1.43. Viscosity (n)=4.65 Pa·s at 25° C., 3.48 Pa·s at 37° C. ¹H NMR (400 MHZ, CDCl3, 25° C.) δ=3.86 (b, 2H, —CO₂CH₂—), 2.10-1.72 (b, 2H, —CH₂—), 1.61 (b, 2H, —CO₂CH₂CH₂—), 1.38-1.25 (b, 4H, —SiCH₂CH₂CH₂CH₃), 1.11-0.78 (b, 6H, —CH₃), 0.60-0.44 (b, 4H, —SiCH₂—), 0.16-0.01 (b, 36H, —Si(CH₃) ₂). A ¹H NMR spectrum of poly (PDMS-MA) tri-arm star bottlebrush polymer in CDCl3 is shown in FIG. 2.

### Example 2

### HEMA Chain Extension

HEMA chain extenders were added to the three-arm star bottlebrush polymers synthesized in Example 1 via RAFT polymerization as shown in Scheme 2, below

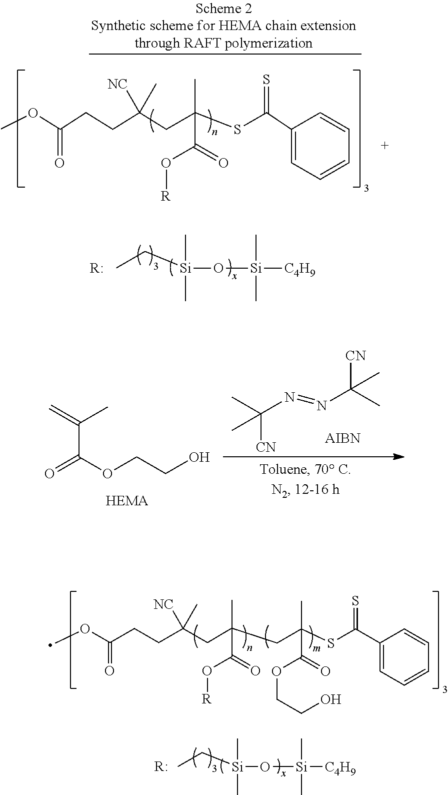

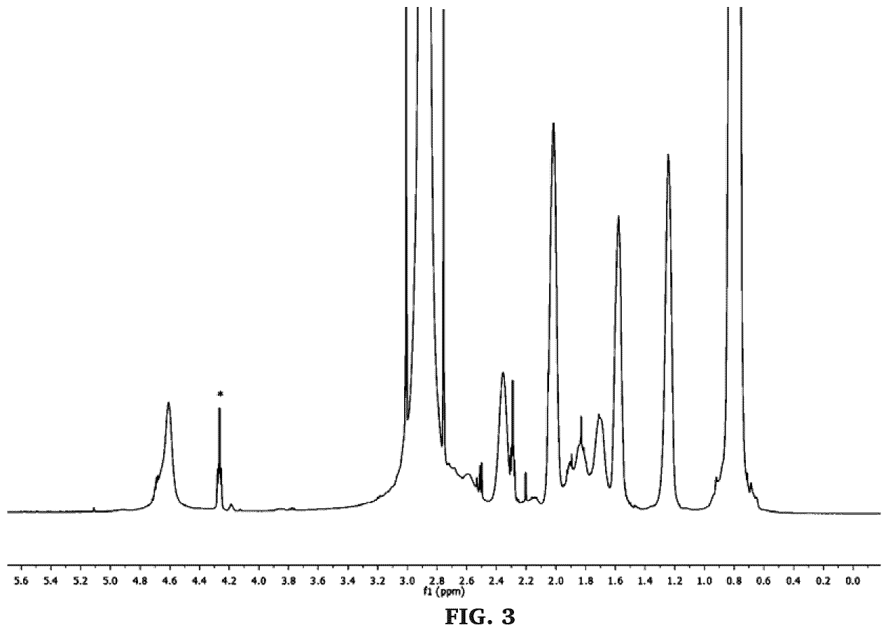

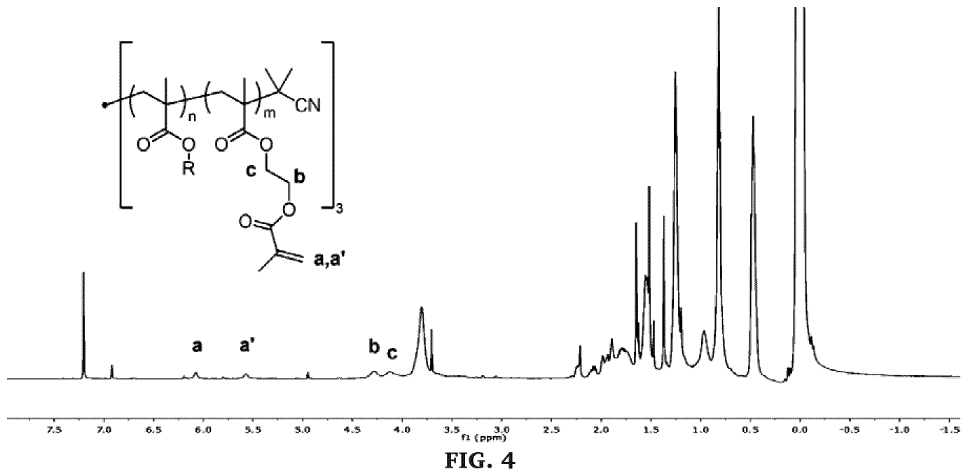

The tri-arm star bottlebrush (BB) polymer synthesized in Example 1 was used as a macro-chain transfer agent (mCTA) to grow a HEMA block on the star polymer. Tri-arm star BB polymer (mCTA, 2.0 g, 0.0281 mmol, 1 equiv) was dissolved in 5.0 mL of toluene first, then AIBN (I, ca. 3.0 mg, 0.0141 mmol, 0.5 equiv) and HEMA (M, 0.10-0.11 mL, 0.8432 mmol, 30 equiv) were added to the Schlenk flask. The reaction flask was equipped with a Teflon coated stir bar and sealed with a rubber septum. The solution was sparged with N₂ for 20-30 min and placed in a pre-heated oil bath at 70° C. The polymerization was run for 12-16 hours. The polymerization was quenched by opening the flask to air and adding 10-15 mL of MeOH or acetone directly to the flask. The resulting mixture was vortexed, sonicated, and placed in an ice bath for a couple of minutes. Then, the top liquid layer was decanted, and this purification step was repeated 2 to 4 more times. The final polymer was dissolved in THE, the solution was passed through a 1 μm PTFE filter, all the volatiles were removed under reduced pressure using a rotovap (typically 85-90 mbar, 35-40° C.), and the resulting highly viscous liquid polymer was dried at high vacuum at room temperature overnight. Yellow colored, transparent polymer was obtained (>95% monomer conversion, 1.4 g isolated yield). ¹H NMR (400 MHz, CDCl3, 25° C.) _{8=3.86} (b, 2H, —CO₂CH₂—), 2.10-1.72 (b, 2H, —CH₂—), 1.61 (b, 2H, —CO₂CH₂CH₂—), 1.38-1.25 (b, 4H, —SiCH₂CH₂CH₂CH₃), 1.11-0.78 (b, 6H, —CH₃), 0.60-0.44 (b, 4H, —SiCH₂—), 0.16-0.01 (b, 36H, —Si(CH₃)₂). A ¹H NMR spectrum of HEMA chain-extended poly(PDMS-MA-co-HEMA) tri-arm star polymer in CDCl3 is shown in FIG. 3.

### Example 3

### End-Group Removal (EGR) and Radical Induced Coupling (Excess AIBN Treatment)

The terminal trithiolcarbonate groups remaining from the tri-functional chain transfer agent were removed and replaced with 2-cyanopropyl end capping groups by AIBN treatment as shown in Schemes 3 and 4, below.

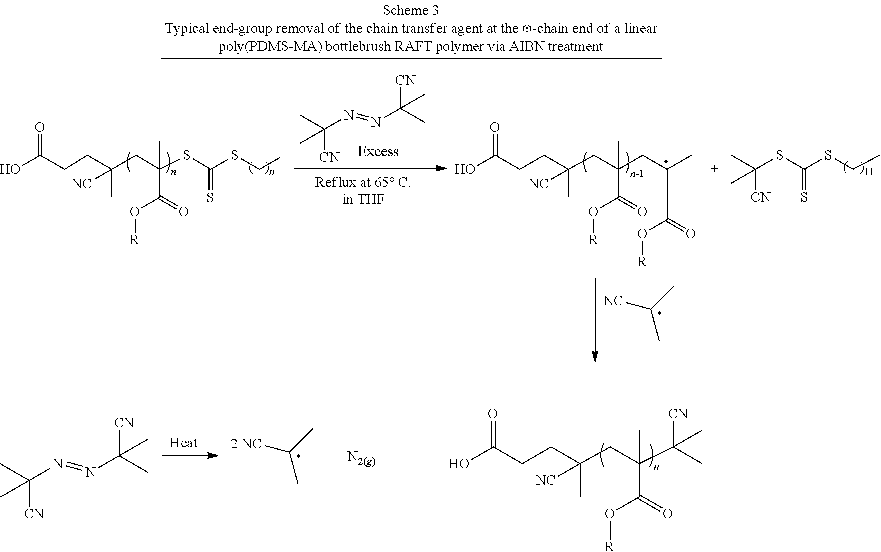

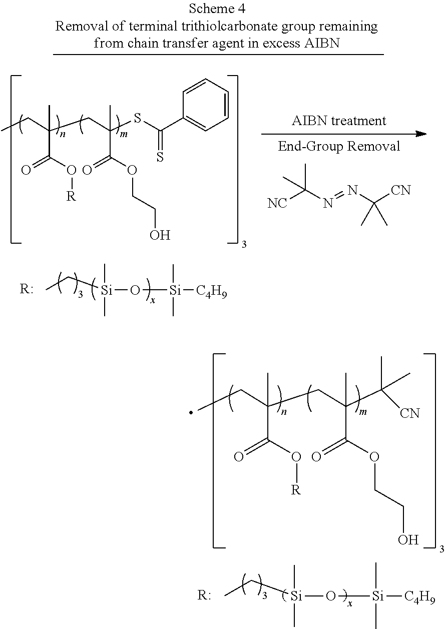

End-group removal was adapted from the literature. In these experiments, chain-transfer agent (CTA) end-groups were removed from polymers using excess AIBN as shown Schemes 3 and 4. First, tri-arm star BB polymer was dissolved in THF (ca. 0.1 g/mL solution), and AIBN was added into the flask (20 equiv relative to CTA used to synthesize the polymer). The flask was equipped with a Teflon coated stir bar and sealed with a rubber septum. The resulting mixture was sparged with N₂ for 20-30 min (>1 mL/min). Then, the flask was submerged into an oil bath and refluxed at 65-70° C. for 5-6 hours (Half-life, tuz, of AIBN at 70° C. is around 5 hours). After the AIBN treatment, the reaction mixture was dried under reduced pressure using a rotovap (typically at 80-100 mbar, 35° C.). The viscous liquid was washed with methanol five times, redissolved in THF, and passed through a 1 μm PTFE filter. Finally, all volatiles were removed (at 80-100 mbar, 35° C.), and the resulting transparent and colorless highly viscous liquid polymer was further dried under high vacuum overnight at room temperature before using for the next step.

### Example 4

### End-Group Functionalization (EGF) of HEMA Chains

The hydroxy groups of the HEMA residues on the chain extended tri-arm star bottlebrush (BB) polymer of Example 3 were functionalized with methacrylic groups as shown in Scheme 5, below.

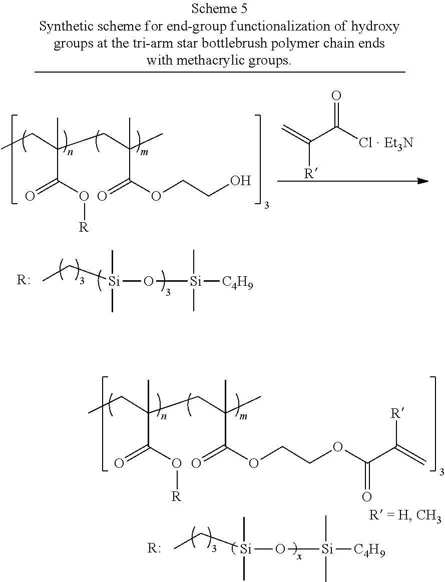

First, the chain-extended tri-arm star BB polymer (ca. 1.4-1.5 g) was dissolved in THE (ca. 100 mL), and the solution was sparged with N₂ for 30-60 min and cooled down to 0° C. in an ice bath. Triethylamine (TEA) was then slowly added to the flask, and the mixture was stirred for 5-10 min. Methacryloyl chloride was added to the flask dropwise, and immediate white precipitate formation was observed. The reaction was let warmed up to the room temperature and run overnight. The reaction mixture was passed through a silica plug using THF as an eluent. Then, all the volatiles were removed under reduced pressure and the resulting crude polymer mixture was washed with methanol (100-200 mL)₃-4 times. A ¹H NMR spectrum of an end-group functionalized tri-arm star bottlebrush polymer in CDCl₃ is shown in FIG. 4.

### Example 5

### Curing Three Arm Star Bottlebrush Resin Under UV Irradiation

The three-arm star bottlebrush of Example 4 was combined with dimethacryloxypropyl terminated polydimethylsiloxane (PDMS-diMA) and a photoinitiator (2,2-dimethoxy-1,2-diphenylethanone) to form a photocrosslinkable Three Arm Star Bottlebrush Resin, as shown in FIG. 5A. In these experiments, a TEFLON™ mold (See, FIG. 5C) having indentations formed to receive the uncured resin and sized to produce elastomers having the general size and shape of an IOL. The uncured resin was poured into the indentations in the mold and then cured by exposure to ultraviolet light (365 nm) to produce soft, flexible, and optically clear hydrogel network comprising the three-arm star bottlebrush polymer as shown in FIGS. 5A and 5B. An infrared spectrum of cured polymer resin showing all the methacrylic functional groups being consumed is shown in FIG. 6.

### Example 6

### Mechanical Analysis of Bottlebrush Elastomers

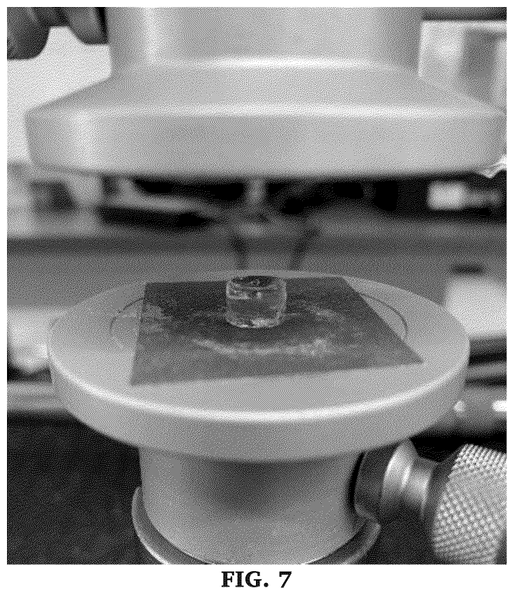

A series of disc shape elastomers with a diameter of ca. 7.80 mm, and a height ranging from 3-5 mm were made from a three-arm star bottlebrush polymer (P(PDMS-MA)-based; DP=30-35/arm) at different PDMS-diMA concentrations and with and without a solvent (ethyl acetate). Compression tests were performed on these disc-shaped elastomers using a 3-5 mm. 10 lbs (ca. 44N) load cell as shown in FIG. 7. The details for each sample, as well as the Young's modulus and Ultimate Compressive Strength (UTS) of each sample, are set forth in Table 1, below.

TABLE 1
Summary of compression test results of the disc shape elastomers.

| | | Young's | Ultimate |
|---|---|---|---|
| | | Modulus | Compressive |
| Sample | Details | (MPa) | Strength (MPa) |
| 1_1 | P(PDMS-MA)-based resin | 0.0108 | — |
| 1_2 | (DP = 30-35/arm), PDMS- | 0.013 | 0.0431 |
| | diMA (2 V %), photoinitiator | | |
| | (PI) | | |
| 2_1 | P(PDMS-MA)-based resin | 0.0072 | 0.00695 |
| 2_2 | (DP = 30-35/arm), PDMS- | 0.0045 | 0.00296 |
| | diMA (2 V %), PI, 10 V % | | |
| | solvent (ethyl acetate) | | |
| 3_1 | P(PDMS-MA)-based resin | 0.0217 | 0.0493 |
| 3_2 | (DP = 30-35/arm), PDMS- | 0.011 | 0.0121 |
| | diMA (2 V %), PI, 20 V % | | |
| | solvent (ethyl acetate) | | |
| 4_1 | P(PDMS-MA)-based resin | 0.0146 | 0.037/0.493 |
| 4_2 | (DP = 30-35/arm), PDMS- | 0.0172 | 0.069 |
| 4_3 | diMA (3 V %), photoinitiator | 0.0080 | 0.177 |
| | (PI) | | |
| 5_1 | P(PDMS-MA)-based resin | 0.0139 | 0.269 |
| 5_2 | (DP = 30-35/arm), PDMS- | 0.010 | 0.047 |
| | diMA (4 V %), photoinitiator | | |
| | (PI) | | |

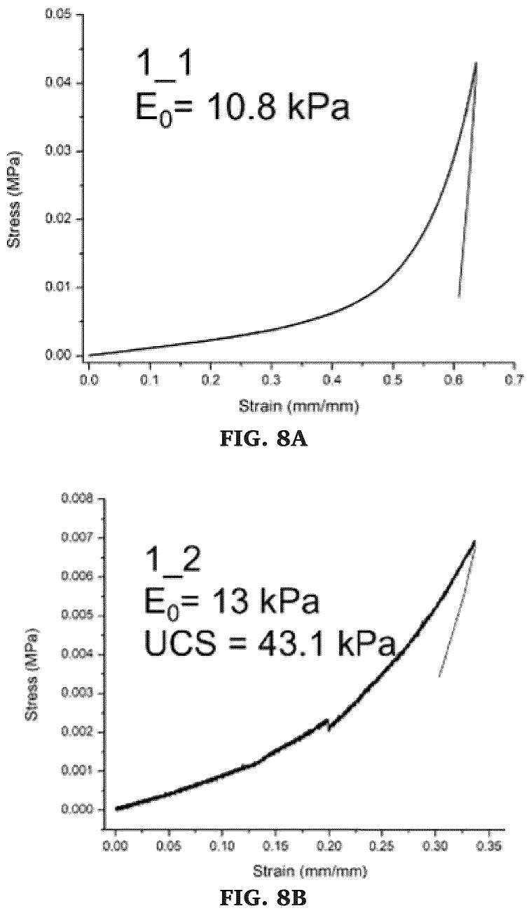

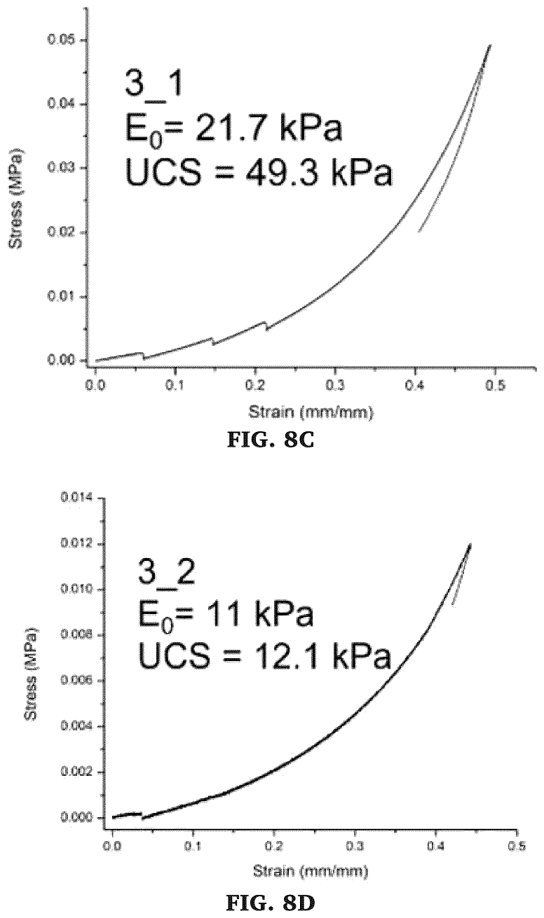

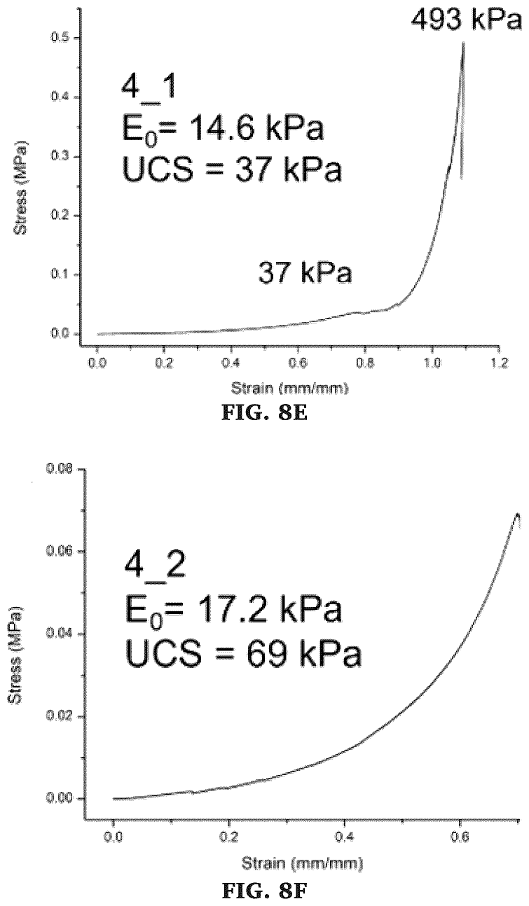

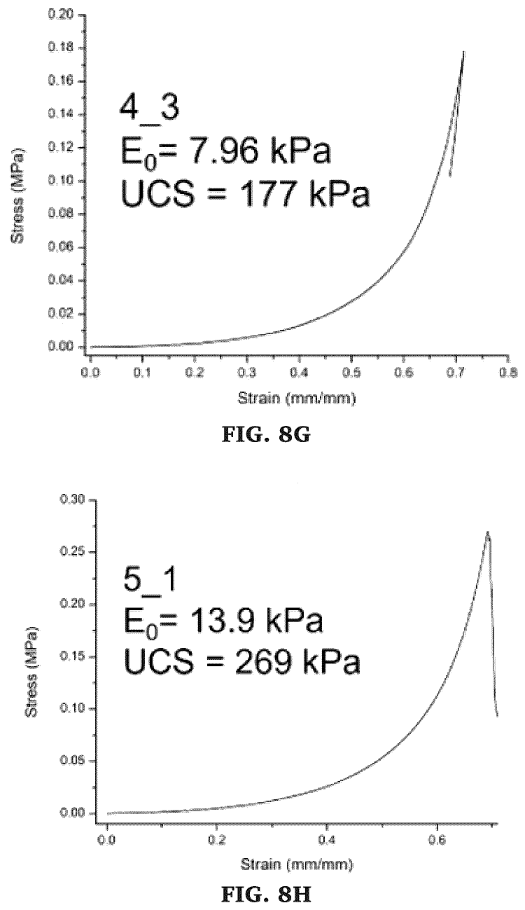

Stress/strain curves for samples 1_1 (FIG. 8A), 1_2 (FIG. 8B), 3_1 (FIG. 8C), 3_2 (FIG. 8D), 4_1 (FIG. 8E), 4_2 (FIG. 8F), 4_3 (FIG. 8G), and 5_1 (FIG. 8H) are shown in FIGS. 8A-H.

In light of the foregoing, it should be appreciated that the present invention significantly advances the art by providing Tri-arm bottlebrush RAFT polymers that are structurally and functionally improved in a number of ways. While particular embodiments of the invention have been disclosed in detail herein, it should be appreciated that the invention is not limited thereto or thereby inasmuch as variations on the invention herein will be readily appreciated by those of ordinary skill in the art. The scope of the invention shall be appreciated from the claims that follow.

What is claimed is:

1. A tri-arm star bottlebrush polymer for use in an intraocular lens comprising the residue of a trifunctional reversible addition fragmentation chain-transfer (RAFT) agent and three methacrylate polymer chains extending therefrom, wherein each one of said three methacrylate polymer chains comprising the polymerized residues of two or more methacrylate macromonomers.

2. The tri-arm star bottlebrush polymer of claim 1 wherein said trifunctional RAFT agent comprises three arms each having at least one site capable of RAFT polymerization, wherein each arm further comprises a sulfur containing end group.

3. The tri-arm star bottlebrush polymer of claim 1 wherein said trifunctional RAFT agent comprises 1,1,1-tris [(dodecylthiocarbonothioylthio)-2-methylpropionate] ethane (Tris (DDMAT).

4. The tri-arm star bottlebrush polymer of claim 1 wherein said two or more methacrylate macromonomers comprise monomethacryloxypropyl terminated polydimethylsiloxane-asymmetric (PDMS-MA).

5. The tri-arm star bottlebrush polymer of claim 1 wherein said methacrylate macromonomers have the formula: 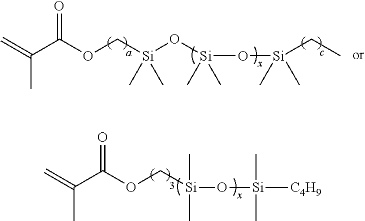
where a is an integer from about 1 to about 6; c is an integer from about 1 to about 5; and
x is an integer from about 2 to about 30.

6. The tri-arm star bottlebrush polymer of claim 3 wherein said PDMS-MA has a mass average molecular weight from about 600 kDa to about 800 kDa.

7. The tri-arm star bottlebrush polymer of claim 1 having the formula: 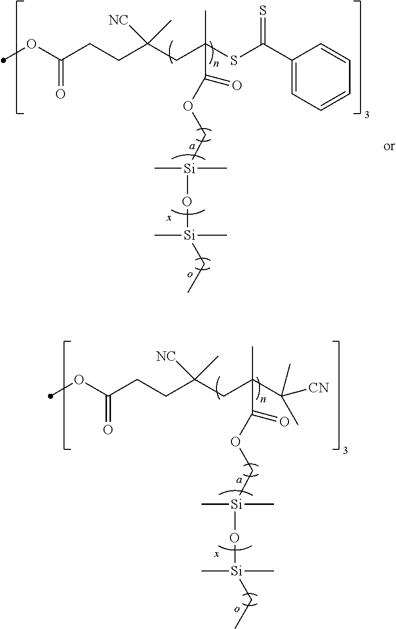
where a is an integer from about 1 to about 6; c is an integer from 1 to 5; x is an integer from about 2 to about 20 and n is an integer from about 10 to about 80.

8. The tri-arm star bottlebrush polymer of claim 1 having the formula: 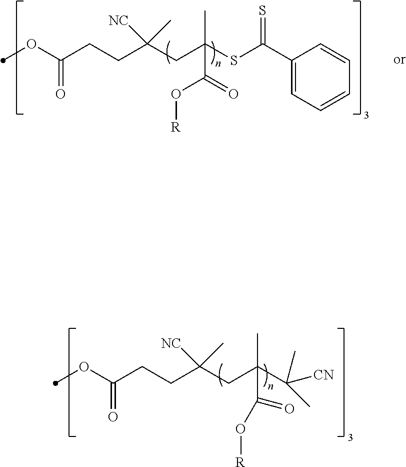
wherein R has the formula 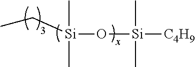
where x is an integer from about 5 to about 10; and n is an integer from about 10 to about 80.

9. The tri-arm star bottlebrush polymer of claim 1 wherein each of said three methacrylate polymer chains further comprises the residues of one or more hydroxy-functionalized methacrylate chain extenders.

10. The tri-arm star bottlebrush polymer of claim 9 wherein said one or more hydroxy-functionalized methacrylate chain extenders is 2-hydroxyethyl methylacrylate (HEMA) molecules.

11. The tri-arm star bottlebrush polymer of claim 8 or 9 having a formula selected from: 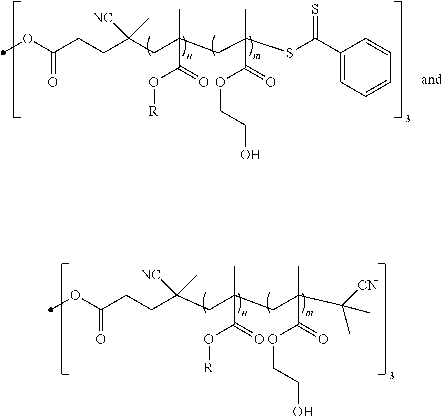
wherein R has the formula 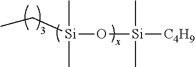
where x is an integer from about 5 to about 10; n is a mole percent from about 80% to about 99%; and m is a mole percent from about 1% to about 20%.

12. The tri-arm star bottlebrush polymer of claim 11 wherein n is a mole percent from about 90% to about 99%.

13. The tri-arm star bottlebrush polymer of claim 11 wherein n is a mole percent from about 95% to about 99%.

14. The tri-arm star bottlebrush polymer of claim 10 wherein each of said three methacrylate polymer chains comprises an A: B block copolymer having a poly(monomethacryloxypropyl terminated polydimethylsiloxane) A block and a poly(2-hydroxyethyl methylacrylate) B block.

15. The tri-arm star bottlebrush polymer of claim 9 or 10 further comprising a plurality of alkene functional groups covalently bonded to said three methacrylate polymer chains through terminal hydroxyl groups on said two or more hydroxy-functionalized methacrylate chain extenders.

16. The tri-arm star bottlebrush polymer of claim 10 further comprising a plurality of alkene functional groups covalently bonded to said three methacrylate polymer chains through terminal hydroxyl groups on said two or more 2-hydroxyethyl methylacrylate (HEMA) molecules.

17. The tri-arm star bottlebrush polymer of claim 15 or 16 having a formula selected from: 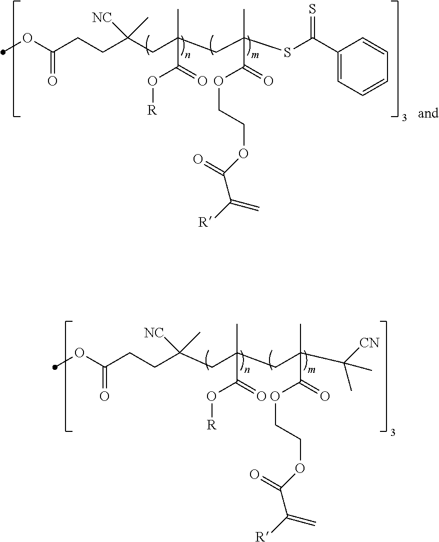
wherein R has the formula 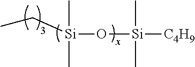
where x is an integer from about 5 to about 10; R′ is H or CH₃; n is a mole percent from about 80% to about 99%; and m is a mole percent from about 1% to about 20%.

18. The tri-arm star bottlebrush polymer of claim 1 or 2, wherein any of said sulfur containing end groups remaining from said trifunctional a reversible addition fragmentation chain-transfer (RAFT) agent after polymerization have been removed.

19. The tri-arm star bottlebrush polymer of claim 9 or 10 wherein any sulfur containing end groups remaining from said trifunctional a reversible addition fragmentation chain-transfer (RAFT) agent after polymerization have been removed.

20. The tri-arm star bottlebrush polymer of claim 15 or 16 wherein any of said sulfur containing end groups remaining from said trifunctional a reversible addition fragmentation chain-transfer (RAFT) agent have been removed.

21. The tri-arm star bottlebrush polymer of claims 18-20 having a formula selected from: 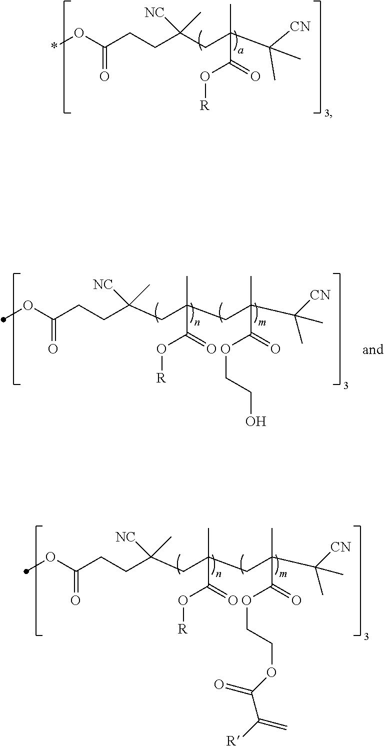
wherein R has the formula 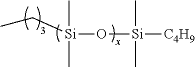
where x is an integer from about 5 to about 10; R′ is H or CH₃; a is an integer from about 10 to about 80; n is a mole percent from about 80% to about 99%; and m is a mole percent from about 1% to about 20%.

22. The tri-arm star bottlebrush polymer of claims 28-21 wherein said tri-arm star bottlebrush polymer is optically clear.

23. The tri-arm star bottlebrush polymer of claim 1 wherein said tri-arm star bottlebrush polymer has a refractive index of from about 1.40 to about 1.49, preferably from about 1.42 to about 1.48, and more preferably from about 1.43 to about 1.46 at 37° C.

24. The tri-arm star bottlebrush polymer of claim 18 or 21 wherein said tri-arm star bottlebrush polymer has a refractive index of from about 1.40 to about 1.49, preferably from about 1.42 to about 1.48, and more preferably from about 1.43 to about 1.46 at 37° C.

25. The tri-arm star bottlebrush polymer of claims 1-24 having a degree of polymerization for each arm between about 10 and about 80.

26. A photocurable tri-arm star bottlebrush polymer resin comprising the tri-arm star bottlebrush polymer of any of claims 1-25, dimethacryloxypropyl terminated polydimethylsiloxane (PDMS-diMA) and a photoinitiator.

27. The photocurable tri-arm star bottlebrush polymer resin of claim 26 comprising from about 2% to about 98% PDMS-diMA by volume.

28. The photocurable tri-arm star bottlebrush polymer resin of claim 26 wherein said photoinitiator is 2,2-dimethoxy-1,2-diphenylethanone.

29. The photocurable tri-arm star bottlebrush polymer resin of claim 26 wherein said resin is optically clear.

30. A soft and flexible tri-arm star bottlebrush hydrogel network for use in artificial intraocular lenses comprising the photocurable tri-arm star bottlebrush polymer resin of claim 26.

31. The soft and flexible photocured tri-arm star bottlebrush hydrogel network of claim 30 having Young's modulus of from about 0.005 MPa to about 0.05 MPa.

32. The soft and flexible photocured tri-arm star bottlebrush hydrogel network of claim 30 having an ultimate compressive strength (UCS) of from about 0.002 MPa to about 0.5 MPa.

33. The soft and flexible photocured tri-arm star bottlebrush hydrogel network of claim 30 wherein said hydrogel network is optically clear.

34. An artificial intraocular lens comprising the tri-arm star bottlebrush polymer of claims 1-25.

35. The artificial intraocular lens of claim 34 having Young's modulus of from about 0.005 MPa to about 0.05 MPa.

36. The artificial intraocular lens of claim 34 having an ultimate compressive strength (UCS) of from about 0.002 MPa to about 0.5 MPa after curing with ultraviolet light.

37. The artificial intraocular lens of claim 34 wherein said artificial intraocular lens is optically clear.

38. A method for making a photocured tri-arm star bottlebrush hydrogel network comprising:
A) combining a tri-arm star bottlebrush polymer of claims 1-31 with a bis-methacryl terminated polydimethylsiloxane crosslinker, and a photoinitiator to form an uncured tri-arm star bottlebrush polymer resin; and
B) exposing the uncured tri-arm star bottlebrush polymer resin to ultraviolet light to produce a photocured tri-arm star bottlebrush hydrogel network.

39. The method of claim 38 wherein the bis-methacryl terminated polydimethylsiloxane crosslinker is dimethacryloxypropyl terminated polydimethylsiloxane (PDMS-diMA.

40. The method of claim 39 wherein said uncured tri-arm star bottlebrush polymer resin comprises from about 2% to about 98% by volume PDMS-diMA.

41. The method of claim 38 wherein said photoinitiator is 2,2-dimethoxy-1,2-diphenylethanone.

42. The method of claim 38 wherein said photocured tri-arm star bottlebrush hydrogel network has a Young's modulus of from about 0.005 MPa to about 0.05 MPa.

43. The method of claim 38 wherein said photocured tri-arm star bottlebrush hydrogel network has an ultimate compressive strength (UCS) of from about 0.002 MPa to about 0.5 MPa.

44. The method of claim 38 wherein said photocured tri-arm star bottlebrush hydrogel network produced is optically clear.

45. A method of making an artificial intraocular lens comprising the tri-arm star bottlebrush polymer of claims 1-25 comprising:
A) preparing a mold shaped to hold an artificial intraocular lens of a desired size and shape;
B) combining a tri-arm star bottlebrush polymer of claims 1-31 with PDMS-diMA, and a photoinitiator to form an uncured tri-arm star bottlebrush polymer resin;
C) filling the mold of step A with the uncured tri-arm star bottlebrush polymer resin of step B; and
D) exposing the uncured tri-arm star bottlebrush polymer resin to ultraviolet light to produce an artificial intraocular lens by crosslinking said uncured tri-arm star bottlebrush polymer resin to form an optically clear photocured tri-arm star bottlebrush hydrogel network.

46. A method of making an artificial intraocular lens comprising the tri-arm star bottlebrush polymer of claims 1-32 comprising:
A) creating a mold for an optic with refractive or diffractive surfaces for use as a presbyopia correcting IOL;
B) combining a tri-arm star bottlebrush polymer of claims 1-31 with PDMS-diMA, and a photoinitiator to form an uncured tri-arm star bottlebrush polymer resin;
C) filling the mold of step A with the uncured tri-arm star bottlebrush polymer resin of step B; and
D) exposing the uncured tri-arm star bottlebrush polymer resin to ultraviolet light to crosslink it and produce an artificial intraocular lens having an ideal optic for a presbyopia correcting IOL and comprising photocured tri-arm star bottlebrush hydrogel network.

47. A method of making an artificial intraocular lens comprising the tri-arm star bottlebrush polymer of claims 1-32 comprising:
A) creating a computer model of the ideal shape of an IOL for a specific patient;
B) using said computer model to generate a mold shaped to said ideal shape;
C) combining a tri-arm star bottlebrush polymer of claims 1-31 with PDMS-diMA, and a photoinitiator to form an uncured tri-arm star bottlebrush polymer resin;
D) filling the mold of step A with the uncured tri-arm star bottlebrush polymer resin of step B; and
E) exposing the uncured tri-arm star bottlebrush polymer resin to ultraviolet light to crosslink it and produce an artificial intraocular lens having an ideal shape for said patient and comprising photocured tri-arm star bottlebrush hydrogel network.
